(19) United States
(12) Patent Application Publication
(10) Pub. No.: US 2007/0009561 A1
(43) Pub. Date: Jan. 11, 2007

(54) Tablets resistant to shock loads

(76) Inventors: Thomas Holderbaum, Hilden (DE); Ulrich Pegelow, Dusseldorf (DE); Christian Nitsch, Dusseldorf (DE); Uta Steffen-Holderbaum, Koln (DE)

Correspondence Address: PAUL & PAUL, 2900 TWO THOUSAND MARKET STREET, PHILADELPHIA, PA, 19103, US

(21) Appl. No.: 11/433,877

(22) Filed: May 12, 2006

Related U.S. Application Data
(63) Continuation of application No. PCT/EP04/10251, filed on Sep. 14, 2004.

(30) Foreign Application Priority Data
Nov. 13, 2003 (DE) DE 103 52 961.6

(51) Int. Cl. A61K 8/02

(52) U.S. Cl. 424/401

(57) ABSTRACT

The invention relates to tablets consisting of pressed particulate washing or cleaning agents and comprising at least two reinforcing grooves on the upper side thereof, the horizontal extension of said grooves on the plane of the tablet surface being longer than the depth thereof. The inventive tablets are characterized by a short disintegration time for a pre-determined hardness and exhibit increased stability in terms of shock loads and falls.

### CROSS-REFERENCE TO RELATED APPLICATIONS

This application is a continuation under 35 U.S.C. §365(c) and 35 U.S.C. §120 of International Application No. PCT/EP2004/010251, filed Sep. 14, 2004. This application also claims priority under 35 U.S.C. §119 of German Patent Application No. 103 52 961.6, filed Nov. 13, 2003. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention lies in the field of compact tablets which have washing- and cleaning-active properties. Such washing and cleaning composition tablets include, for example, washing composition tablets for the washing of textiles, cleaning composition tablets for machine dishwashing or the cleaning of hard surfaces, bleach tablets for use in washing machines or machine dishwashers, water softening tablets or stain removal tablets. In particular, the invention relates to washing and cleaning composition tablets which are used for cleaning dishware in a domestic machine dishwasher and are referred to for short as cleaning composition tablets or dishwasher detergent tablets.

Washing and cleaning composition tablets are widely described in the prior art and are enjoying increasing popularity with the consumer owing to the simple dosage. Tableted washing and cleaning compositions have a series of advantages over pulverulent washing and cleaning compositions: they are simpler to dose and to handle and, owing to their compact structure, have advantages in storage and in transport. The patent literature too consequently describes washing and cleaning composition tablets comprehensively. One problem which occurs time and again in the case of use of washing- and cleaning-active tablets is the excessively slow decomposition and dissolution rate of the tablets under use conditions. Since sufficiently stable, i.e., dimensionally stable and fracture-resistant, tablets can be produced only by comparatively high compressive pressures, there is high compaction of the tablet constituents and a resulting retarded disintegration of the tablet in the aqueous liquor and hence an excessively slow release of the active substances in the washing or cleaning cycle.

A further problem which occurs especially in the case of washing and cleaning composition tablets is the often insufficient stability of these tablets against the stresses in the course of packaging, transport and handling, i.e. against dropping and impact stresses. After the compression, the tablets are sent on transport belts to the packaging, the tablets being encased with a film individually or in groups and subsequently packaged into cartons. As it is transferred, the tablet in an approximately parabolic trajectory from the conveyor belt of the film wrapping machine, hits the box wall or the tablets already filled beforehand. Here, especially in the case of rectangular tablets, forces occur in longitudinal direction of the tablet and can lead to edge breakage and attrition phenomena and impair the appearance of the tablet or even lead to complete destruction of the tablet structure.

To overcome the dichotomy between hardness, i.e. transport and handling stability, and easy decomposition of the tablets, many approaches to solutions have been developed in the prior art. An approach which is known, in particular, from pharmacy and has expanded to the field of washing and cleaning composition tablets is the incorporation of certain disintegration assistants which ease the ingress of water or swell or evolve gas on ingress of water or have another form of disintegrating action. Other approaches to solutions from the patent literature describe the compression of premixtures of certain particle sizes, the separation of individual ingredients from the certain other ingredients, and the coating of individual ingredients or the entire tablet with binders.

(2) Description of Related Art, Including Information Disclosed Under 37 C.F.R. §§ 1.97 and 1.98.

For instance, EP 687 464 (Allphamed Arzneimittel-Gesellschaft) describes effervescent tablets consisting of at least one active ingredient or an active ingredient combination, at least one binder, optionally carriers such as aromas, dyes, fragrances, plasticizers, bleaches and effervescent additives, the binders used being propylene glycol or glycerol, preferably in amounts of from 0.004 to 2.5% by weight. Likewise claimed are processes for producing these effervescent tablets. According to the statements of this document, it is also possible with the inventive teaching to produce an effervescent washing composition tablet without the binders used leading to loss of carbon dioxide in the effervescent additives.

The European patent application EP 711 828 (Unilever) describes detergent tablets which comprise surfactant(s), builder(s) and a polymer which acts as a binding and disintegration assistant. The binders disclosed in this document are intended to be solid at room temperature and to be added to the premixtures to be compressed as a melt. Preferred binders are the high molecular weight polyethylene glycols.

The use of solid polyethylene glycols is also described in the German patent application DE 197 09 411.2 (Henkel). This document teaches synergistic effects between the polyethylene glycols and overdried amorphous silicates.

Solutions to the problem of friability and attrition stability of washing and cleaning composition tablets are disclosed, for example, in DE 198 41 146 (Henkel). According to the teaching of this document, the addition of nonsurfactant, water-soluble, liquid binders to the mixtures to be tableted has a positive effect on their attrition behavior.

A common feature of all approaches to solutions is the idea of changing the ingredients in a controlled manner or incorporating specific ingredients so as to positively influence the tablet properties. A formulation-independent approach to a solution for the problems mentioned is not disclosed in the prior art.

### BRIEF SUMMARY OF THE INVENTION

It is thus an object of the present invention to provide washing or cleaning composition tablets which, at a given hardness, are notable for short disintegration times and can thus also be metered via the detergent drawer of domestic washing machines. In addition to these requirements, the tablets should have increased stability against impact and dropping stresses. The corresponding advantages should be achieved irrespective of the formulation, in order to be able to dispense with complicated prefinishing steps or the use of expensive tableting assistants just for this purpose.

It has now been found that the surface configuration of the tablets is of particular significance in the solution of the problems mentioned above.

The present invention provides a tablet of compressed particulate washing or cleaning composition, characterized in that it has, on its upper side, at least two reinforcement depressions whose horizontal dimension at the level of the tablet surface is greater than their depth.

An inventive tablet has an upper side and a lower side and one or more side surfaces. The lower side is the surface of the tablets which comes into contact with the lower punch of the tableting press in the compression operation, while the upper side is that surface which contacts the upper punch of the tableting press. The side surfaces are contacted by the walls of the die in the compression operation, a round or oval tablet having only one side wall (the cylindrical jacket), while polygonal tablets have a number of side surfaces equal to the number of corners. Preference is given in accordance with the invention to rectangular tablets which have four side surfaces. In the specific case of the square tablet, all four side surfaces are of equal size, while only two side surfaces in each case are the same in tablets with rectangular upper side and lower side.

### BRIEF DESCRIPTION OF THE SEVERAL VIEWS OF THE DRAWING(S)

Not Applicable

### DETAILED DESCRIPTION OF THE INVENTION

According to the invention, the upper side of the tablet is provided with reinforcement depressions which are molded into the upper side. The depressions on the upper side of the tablet correspond to elevations on the tableting punch (see below). According to the invention, the horizontal dimension of the reinforcement depressions at the level of the tablet surface is greater than their depth. In other words, the reinforcement depressions have, in x,y direction at the level of the upper side of the tablet, a greater dimension than in z direction of the tablet height. In tablets preferred in accordance with the invention, the horizontal dimension of the reinforcement depressions at the level of the tablet surface is from 1.01 times to 5 times, preferably from 1.02 times to 4 times, more preferably from 1.04 times to 3 times and, in particular, from 1.05 times to 2 times the depth of the reinforcement depressions.

A 5 mm-deep reinforcement depression accordingly has a length or width of more than 5 mm, the length or width in preferred tablets being from 5.05 to 25 mm, preferably from 5.5 to 20 mm, more preferably from 5.2 to 15 mm and, in particular, from 5.25 to 10 mm.

In tablets preferred in accordance with the invention, the depth of the reinforcement depressions correlates with the height of the tablets in order to further optimize the fracture stability (breaking stability) of the tablet. Here, particularly preferred inventive tablets are characterized in that the depth of the reinforcement depressions is from 0.05 times to 0.5 times, preferably from 0.1 times to 0.4 times and, in particular, from 0.15 times to 0.3 times the tablet height.

According to the invention, the tablets have at least two reinforcement depressions. Depending on the form of the reinforcement depressions, it is, however, also possible for more than two depressions to be provided on the upper side of the tablet. For example, preference is given to inventive tablets which have at least 3, preferably at least 4, particularly preferably at least 5, more preferably at least 6, more preferably at least 7, more preferably at least 8, more preferably at least 9 and, in particular, at least 10 reinforcement depressions.

The reinforcing depressions may be molded into the tablet surface as straight grooves, but they may also be angled or wavy lines or closed outlined figures. In the simplest case, the reinforcement depressions molded in accordance with the invention are grooves, i.e. straight lines which run parallel to one another on the surface of the tablet and join one side of the tablet surface to the opposite side. These angles enclose an angle with the lateral delimiting line of the tablet surface. When this angle is 90°, the reinforcement depressions run parallel to the tablet width or length. Angles of <90° can be realized by virtue of the reinforcement depressions running obliquely over the tablet surface. Preferred inventive tablets are characterized in that the reinforcement depressions run parallel to one another and to the tablet width, preference being given to an equidistant arrangement of the reinforcement depressions.

This parallel arrangement can be realized not only with straight lines but also with reinforcement depressions configured in a curved or wavelike manner. It is also possible to combine mutually parallel reinforcement depressions which run parallel to the tablet width with further mutually parallel reinforcement depressions which run parallel to the tablet length. This “waffle iron structure” in which the crossing reinforcement depressions enclose an angle of 90° with one another can of course also be modified to the effect that the crossing angle is changed. In that case, at least one series of mutually parallel reinforcement depressions does not run parallel to the tablet width or length. Preference is given in accordance with the invention to inventive tablets in which the reinforcement depressions run parallel to the tablet width and further reinforcement depressions run parallel to the tablet length, preference being given to an equidistant arrangement of the reinforcement depressions.

In inventive tablets, a further preferred arrangement of the reinforcement depressions consists in the reinforcement depressions starting from a common center in a radiating manner. This arrangement is particularly advantageous, for example, when the tablet has further depressions which are utilized as a depression or cavity in order to incorporate other ingredients therein or to insert other tablet constituents. Such depression tablets are currently known as dishwasher detergents in the prior art and can be stabilized by the inventive reinforcement depressions. Visually, such an arrangement of reinforcement depressions is reminiscent of the sun with the corresponding number of rays, for example, four, five, six, seven or eight rays. It will be appreciated that the “sun rays” can also be formed not just by straight lines but also by curved or wavy lines.

A further means of arranging the reinforcement depressions consists in configuring them in outline form. Preference is given here to inventive tablets in which the reinforcement depressions have the form of concentric circles or ellipses. Also useful are more complicated forms such as clouds, trees, cups, hands, etc.

Irrespective of the outward form of the reinforcement depressions, they preferably have a cross section which is semicircular or semielliptical or triangular. The “cross section” of the reinforcement depressions is the square cut through the tablet, vertically to the particular reinforcement depression. Accordingly, preference is given to inventive tablets in which the cross section of the reinforcement depressions is triangular or semicircular.

As already mentioned above, the tablet height and the depth of the reinforcement depressions preferably correlate to one another. In addition to the relative statements mentioned above, it is also possible to make absolute statements on particularly advantageous embodiments. Preferred inventive tablets are characterized in that the height of the tablet is from 5 to 25 mm, preferably from 7 to 22 mm and, in particular, from 10 to 20 mm. Accordingly, the depth of the reinforcement depressions in preferred inventive tablets is from 0.5 to 10 mm, preferably from 0.75 to 8 mm and, in particular, from 1 to 5 mm. The depth of the reinforcement depressions is the lowest point of the particular reinforcement depressions, for example, the point of the triangle in the case of V-shaped reinforcement depressions.

To produce the inventive tablets, particulate premixtures are compacted in a die between two punches to form a solid compact. This operation, which is referred to below as tableting for short, is divided into four sections: metering, compaction (elastic reshaping), plastic reshaping and expulsion.

First, the premixture is introduced into the die, the fill level and thus the weight and the shape of the resulting tablet being determined by the position of the lower punch and the shape of the compression tool. Even in the case of high tablet throughputs, the uniform metering is preferably achieved by volumetric metering of the premixture. In the further course of tableting, the upper punch contacts the premixture and descends further in the direction of the lower punch. In the course of this compaction, the particles of the premixture are pressed closer to one another, in the course of which the depression volume within the filling between the punches decreases continuously. From a certain position of the upper punch (and thus from a certain pressure on the premixture), plastic reshaping begins, in the course of which the particles coalesce and the tablet is formed. Depending on the physical properties of the premixture, a portion of the premixture particles is also crushed and there is sintering of the premixture at even higher pressures. At increasing compaction rate, i.e. high throughput amounts, the phase of elastic reshaping is shortened ever further, so that the resulting tablets can have cavities of greater or lesser size. In the last step of the tableting, the finished tablet is pushed out of the die by the lower punch and conveyed away by downstream transport devices. At this time, only the weight of the tablet has been ultimately defined, since the compacts may still change their shape and size owing to physical processes (elastic relaxation, crystallographic effects, cooling, etc.).

The tableting is effected in customary tableting presses which may in principle be equipped with single or double punches. In the latter case, not only the upper punch is used for pressure buildup; the lower punch also moves toward the upper punch during the compaction operation, while the upper punch presses downward. For small production amounts, preference is given to using eccentric tableting presses in which the punch(es) is/are secured to an eccentric disc which is in turn mounted on an axle having a particular rotation rate. The movement of these compression punches is comparable to the way in which a typical four-stroke engine works. The compaction can be effected with one upper and one lower punch, but a plurality of punches may also be secured to one eccentric disc, in which case the number of die bores is increased correspondingly. The throughputs of eccentric presses vary by type from a few hundred to a maximum of 3,000 tablets per hour.

In eccentric presses, the lower punch is generally not moved during the pressing operation. A consequence of this is that the resulting tablet has a hardness gradient, i.e. is harder in the regions which are closer to the upper punch than in the regions which are closer to the lower punch.

For greater throughputs, rotary tableting presses are selected, in which a greater number of dies is arranged in a circle on what is known as a die table. The number of dies varies by model between 6 and 55, larger dies also being commercially available. An upper and lower punch is assigned to each die on the die table, and the compression pressure can again be built up actively only by the upper or lower punch, or else by both punches. The die table and the punches move about a common vertical axis, the punches being brought into the positions for filling, compaction, plastic reshaping and expulsion with the aid of rail-like cam tracks during the rotation. At the points at which particularly severe raising or lowering of the punches is required (filling, compaction, expulsion), these cam tracks are supported by additional low-pressure sections, low-tension rails and discharge tracks. The dies are filled via a rigidly mounted feed apparatus, known as the filling shoe, which is connected to a stock vessel for the premixture. The compression pressure on the premixture can be adjusted individually via the compression paths for upper and lower punch, in which case the pressure is built up by virtue of the rolling movement of the punch shaft heads past adjustable pressure rolls.

To increase the throughput, rotary presses may also be provided with two filling shoes, in which case only one half-circle has to be passed through to produce one tablet. To produce two-layer and multilayer tablets, a plurality of filling shoes are arranged in series, without the lightly pressed first layer being expelled before the further filling. Suitable process control makes it possible in this way also to produce coated tablets and inlay tablets which have an onion-like structure, the top face of the core or of the core layers in the case of the inlay tablets not being covered and thus remaining visible. Rotary tableting presses can also be equipped with single or multiple tools, so that, for example, an outer circle having 50 bores and an inner circle having 35 bores may be utilized simultaneously for compression. The throughputs of modern rotary tableting presses are more than one million tablets per hour.

It will be appreciated that the tablets, in the context of the present invention, can likewise be configured in multiphase, especially multilayer, form. The tablets may be manufactured in predetermined three-dimensional shape and predetermined size. Useful three-dimensional shapes include virtually all practicable configurations, i.e., for example, the design in bar, rod or ingot form, cubes, cuboids and corresponding three-dimensional elements having planar side faces, and, in particular, cylindrical embodiments with circular or oval cross section. This last configuration includes the presentation form from the tablet up to compact cylinders having a ratio of height to diameter above 1.

The dimensions of the three-dimensional shape of another embodiment of the tablets are adjusted to the detergent drawer of customary domestic washing machines or the dispenser drawer of commercial machine dishwashers, so that the tablets can be dosed directly into the detergent drawer without dosage assistants, where they dissolve during the rinse-in operation or from where they are released during the cleaning cycle. However, it will be appreciated that it is also possible to use the washing and cleaning composition tablets by means of metering assistants.

After the compression, the washing and cleaning composition tablets have a high stability. The fracture resistance of cylindrical tablets can be calculated by means of the parameter of diametral fracture stress. This can be determined by

$$σ = \frac{2 P}{π Dt}$$

In this formula, σ is the diametral fracture stress (DFS) in Pa, P is the force in N which leads to the pressure exerted on the tablet and causing the fracture of the tablet, D is the tablet diameter in meters and t is the height of the tablets.

In order to obtain inventive tablets, the upper punch of the tableting press used for the production has to have elevations corresponding to the reinforcement depressions molded in the later upper side of the tablet. These elevations are preferably manufactured from wear-resistant materials in order to increase the lifetimes of the tableting punches. Suitable materials have been found to be especially metals and plastics.

The present invention therefore further provides a process for producing tablets of compressed particulate washing or cleaning composition by conventional compaction of particulate premixes, wherein the compression is effected by using an upper punch which has, on its pressing surface, at least two elevations for pressing of reinforcement depressions, whose horizontal dimension at the level of the pressing surface is greater than their height.

The usually pulverulent or finely particulate material to be compressed to tablets is, when particularly complex and production-hindering precautions for a specific distribution are not taken, distributed approximately uniformly in the compression die as it is charged. This has the consequence that the material, at the sites at which the profile of the molding element has the highest elevations, has to be compressed to the greatest extent. Even though the material to be compressed attempts to avoid the highest pressure peaks by a movement in the direction of the less highly stressed regions, the highest specific surface pressures occur in the regions of the highest profile elevations.

When the profile of the molding element consists of a planar surface, for example, the base surface which surrounds the elevations for the reinforcement depressions, the highest surface loadings are to be expected at the elevations, and in the tip or the highest point therein. In the region of the tip, the surface has only quite small angles of inclination in relation to the base surface plane. By definition, these angles of inclination increase in the direction toward the base of the elevations and are at the greatest at the transition into the surrounding base surface. The compressive force acts at right angles to the base surface plane and to the surface element in the center of the tip. With increasing distance from this center element, the compressive force is directed onto an increasingly inclined surface, so that the compressive force is divided into a correspondingly decreasing force component at right angles to the particular surface element and a force component directed in turn at right angles thereto. These transverse forces act quasi-tangentially. The force components at right angles to the normal force are a measure of the shear and abrasion forces which act on the interface between the elevations for the reinforcement depressions and material to be compressed. Owing to these abrasion forces among others, the elevations for the reinforcement depressions have to be produced from a very hard, incompressible material.

The adhesion tendency of the material to be compressed on the surface of the compression punch is determined by factors including the specific surface forces between the material to be compressed and the punch surface, and also by the surface structure. When the surface of the compression or tableting punch has, for example, friction-reducing or lubricating or slide-promoting properties, this prevents or at least reduces the adhesion tendency.

As already mentioned, the compressive forces are directed at right angles to the planar base surface. Since the planar base surface constitutes the lowest height in the profile of the molding element, the lowest compression of the material to be compressed is in this region. This leads to lower surface forces also being expected in the region of the base surface than in the region of the upwardly curved elevations for the reinforcement depressions. For these reasons, the material of the base surface also does not have to be incompressible, especially because only normal forces are expected from the pressure geometry.

Although the structure of beds of pulverulent or finely crystalline substances can be considered to be uniform in relation to relatively large surfaces or volumes, it is quite different in the micro range. As a result of these different density ratios in the micro range, the uniform compressive forces arising at the surface of the base surface material are counteracted by different resistances of the material to be compressed. This leads to the occurrence, at points on the surface spaced apart in the micro range, of different specific pressures and accordingly, in the case of compressible material of the base surface element, very slightly different deformations of the material. This phenomenon, referred to here as flexing, has the consequence of the development of different normal and transverse forces at the material surface, as a result of which the tendency for materials to adhere to the surface of the molding element in the region of the base surface is prevented or at least very substantially reduced.

A tableting punch whose molding element is designed in the form described is advantageously adhesion-preventing or at least adhesion-reducing. Such a pressing tool can be used to achieve long tool lifetimes and impeccable tablet surfaces.

In one embodiment in which the molding element of the tableting punch is not to be delimited laterally by the base surface and the base surface is surrounded by a substantially uniform, incompressible edge strip, knock-on effects of the compression and deformation operation at the inner die wall on the compressible base surface are ruled out. An increasing slope of the edge strip in the upward direction brings about, in an advantageous manner, a clean material distribution in the die and a stabilization of the tablet structure.

Very particular advantages in the production of the tableting punch and the lifetime of the compression tool are achieved in one embodiment in which the molding element consists of a plurality of individual parts. Appropriately, extent and tailoring of the individual parts are oriented to the different materials and material requirements. Thus, the individual manufacture of the elevations for the reinforcement depressions of incompressible material with adhesion-resistant coating at least on the outer surface, of a plate-like element of flexible material for the base surface and of an annular element of incompressible material for the edge strip is an advantageous delimitation for the configuration of the individual parts, which is possible owing to their different materials.

As described above, the coating of the elevations for the reinforcement depressions at the same time has to be hard and resistant toward high surface stresses but additionally also has to have a friction-reducing or lubricating property. For this purpose, nickel-containing surface coatings in which ultrafine PTFE particles (Teflon) are enclosed have been found to be very suitable. These impart to the coating adhesion-preventing and material corrosion-preventing properties. As an alternative, another embodiment found to be useful for the adhesion-reducing coating is one in which the base coating material consists not of nickel but of a nickel-phosphorus alloy.

As a further alternative for the surface coating with at least adhesion-reducing action, but which also otherwise fulfills the requirements for hardness and stability, has been found to be a coating of graphite containing diamond particles. In this case, the elevations for the reinforcement depressions is coated with a graphite layer which is known to be lubricating or slide-promoting, and which serves here simultaneously as a binder for the fixing of diamond particles which themselves impart the required hardness to the surface. Experiments with these surface coatings of the elevations for the reinforcement depressions have shown that, even with very long lifetimes of the tools, no material adhesions were to be observed. It is therefore preferred that the at least adhesion-reducing coating consists substantially of carbon.

The adhesion-preventing or at least adhesion-reducing action of the flexible material for the formation of the base surface has been described above. In experiments, it has been found that, for example, very good results were achievable with the polyurethane material Vulkollan or the PVC material Mipolam. Over use times of several thousand pressings, no adhesions whatsoever to the base surface material were found.

As an alternative to metals which, in some cases, need to be coated, the materials used for the elevations in the upper punch which mold the reinforcement depressions can also be plastic. The manufacture of the elevations from plastic materials makes it possible to produce tableting punches which also realize complicated geometries.

In the context of the present invention, the term “plastics” characterizes materials whose essential constituents consist of macromolecular organic compounds which are formed synthetically or by modification of natural products. In many cases, they are meltable and shapable under certain conditions (heat and pressure). Plastics are thus in principle organic polymers and can be classified either by their physical properties (thermoplastics, thermosets and elastomers), by the type of reaction of their preparation (polymers, polycondensates and polyadducts) or by their chemical nature (polyolefins, polyesters, polyamides, polyurethanes, etc).

In the context of the present invention, the elevations in the upper punch which mold the reinforcement depressions constitute elevations on the molding element of the tableting punch. The surface on which the elevations in the upper punch which mold the reinforcement depressions are mounted may likewise have different forms, a multitude of possibilities being conceivable from the planar, flat surface up to hemispherical configurations. In the context of the present invention, it is preferred on the one hand that the surface on which the elevations in the upper punch which mold the reinforcement depressions reside is planar, i.e. flat.

The base surface on which the elevations in the upper punch which mold the reinforcement depressions reside is preferably also manufactured from plastic, so that preference is given to tableting punches in which the elevations in the upper punch which mold the reinforcement depressions and the flat base surface are manufactured from plastic.

It is particularly preferred in the context of the present invention when the material of the elevations in the upper punch which mold the reinforcement depressions is harder than that of the base surface.

In the context of the present invention, the term “hardness” is the term for the resistance offered by a solid body to the penetration of another body. While, for example, the so-called scratch hardness (Mohs hardness) is measured for minerals, other methods for hardness testing have become established in industry. Most frequently employed in this context are Brinell, Rockwell and Vickers methods (particularly for steel and other metals). To determine the Brinell hardness (HB, ball pressure hardness, DIN 50351), standardized steel or Widia balls with diameter 10 mm and a test load P (expressed in N) are pressed without impact into the substances to be tested and the surface area O (in mm²) of the impressed dome of diameter d is determined. The Brinell hardness is then given by:

$$HB = \frac{P}{O} = \frac{1}{9.817 π} \cdot \frac{2 P}{D \cdot ( D - \sqrt{{D}^{2} - {d}^{2}} )}$$

In the case of the measurement, suitable for higher hardness levels, of the Rockwell hardness (HR), either a diamond cone (HRC) or steel balls of various diameters (HRB) are pressed into the material. In the case of the determination of the Vickers hardness (HV), a diamond pyramid with an opening angle between surfaces of 136° is used; here too, the hardness is defined as the load based on the indentation surface (N/mm²). In this test method, the indentations are very small, so that it is also possible to determine the hardness for very thin layers. This also applies analogously to the Knoop hardness (HK) which is determined by using a diamond pyramid with a rhombic base. In the impact hardness determination, the basis for calculation used is the diameter of a sphere indentation which has been obtained by impact with a hand-held hammer (Poldi hammer, scleroscope) or by a tensioned spring. Another, likewise dynamic method for hardness determination is the rebound method. The Shore hardness determined in this way is determined as the rebound hardness by the falling ball test in the case of steel, or as the penetration resistance relative to a frustum in the case of rubber and other elastomers.

In the case of harder plastics, for example, in the case of hard thermoplastics and particularly in the case of thermosets, the ball pressure hardness is measured as the quotient of test force and surface area of the indentation of a steel ball (diameter 5 mm) after 10, 30 or 60 seconds under load.

As described above, the elevations in the upper punch which mold the reinforcement depressions consist of a harder plastic than the base surface. Hard plastics fulfill, in particular, the requirement profile for the elevations in the upper punch which mold the reinforcement depressions at the same having to be hard and resistant against high su face stresses but secondly also having to have a friction-reducing or lubricating property.

Plastics materials which have been found to be appropriate for the elevations in the upper punch which mold the reinforcement depressions are, in particular, polyolefins, preferably polyethylene or polypropylene. Polyethylenes (PE) are polyolefin polymers containing groups of the type
—[CH₂—CH₂]—
as the characteristic base unit of the polymer chain. Polyethylenes are prepared by addition polymerization of ethylene in accordance with two fundamentally different methods, the high-pressure process and the low-pressure process. The resulting products are often referred to accordingly as high-pressure polyethylene and low-pressure polyethylene respectively; they differ primarily in terms of their degree of branching and, associated with this, in their degree of crystallinity and their density. Both processes may be conducted as solution polymerization, emulsion polymerization or gas phase polymerization processes.

In the case of the high-pressure process, branched polyethylenes of low density (approximately 0.915-0.935 g/cm³) and degrees of crystallinity of approximately 40-50% are obtained, which are referred to as LDPE grades. Products with higher molecular mass and consequently improved strength and stretchability bear the abbreviated designation HMW-LDPE (HMW=high molecular weight). By copolymerization of ethylene with longer-chain olefins, especially with butene and octene, it is possible to reduce the marked degree of branching of the polyethylenes produced in the high-pressure process; the copolymers have the abbreviation LLD-PE (linear low density polyethylene).

The macromolecules of the polyethylenes from low pressure processes are substantially linear and unbranched. These polyethylenes (HDPE) have degrees of crystallinity of 60-80% and a density of approximately 0.94-0.965 g/cm³. They are particularly suitable as materials for the elevations in the upper punch which mold the reinforcement depressions.

Polypropylenes (PP) are thermoplastic polymers of propylene comprising base units of the type
—[CH(CH₃)—CH₂]—

Polypropylenes may be prepared by stereospecific polymerization of propylene in the gas phase or in suspension to give highly crystalline isotactic polypropylenes or less crystalline syndiotactic polypropylenes or amorphous atactic polypropylenes. Of particular importance in industry is isotactic polypropylene, in which all methyl groups are located on one side of the polymer chain. Polypropylene is notable for high hardness, resilience, stiffness and thermal stability and is therefore an ideal material for the elevations in the upper punch which mold the reinforcement depressions in the context of the present invention.

An improvement in the mechanical properties of the polypropylenes can be achieved by reinforcement with talc, chalk, wood flour or glass fibers, and the application of metallic coatings is also possible.

In addition to the polyolefins, polyamides are materials usable with preference in the context of the present invention for the elevations in the upper punch which mold the reinforcement depressions. Polyamides are high molecular weight compounds comprising units linked by peptide bonds. With few exceptions, the synthetic polyamides (PA) are thermoplastic, chainlike polymers with repeating acid amide moieties in the main chain. According to chemical structure, the so-called homopolyamides may be divided into two groups: the aminocarboxylic acid types (AS) and the diamine-dicarboxylic acid types (AA-SS); A denotes amino groups and S carboxyl groups. The former are formed from a building block by polycondensation (amino acid) or addition polymerization (w-lactam), the latter from two building blocks by polycondensation (diamine and dicarboxylic acid).

The polyamides comprising unbranched aliphatic building blocks are coded according to the number of carbon atoms. For example, the designation PA 6 is the polyamide constructed from ε-aminocaproic acid or ε-caprolactam. PA 12 is a poly(ε-laurolactam) from ε-laurolactam. In the case of the AA-SS type, first the carbon number of the diamine and then that of the dicarboxylic acid are stated: PA 66 (polyhexamethyleneadipamide) is formed from hexamethylenediamine (1,6-hexanediamine) and adipic acid, PA 610 (polyhexamethylenesebacamide) from 1,6-hexanediamine and sebacic acid, PA 612 (polyhexamethylenedodecanamide) from 1,6-hexanediamine and dodecanedioic acid. The polyamide types mentioned are materials preferred in the context of the present invention for the elevations in the upper punch which mold the reinforcement depressions.

Polyurethanes (PU) are polymers (polyadducts) obtainable by polyaddition from dihydric and higher polyhydric alcohols and isocyanates, containing groups of type
—[CO—NH—R²—NH—CO—O—R¹—O]—
as characteristic basic units of the base macromolecules, in which R¹ is a low molecular mass or polymeric diol radical and R² is an aliphatic or aromatic group. Industrially important PUs are produced from polyesterdiols and/or polyetherdiols and, for example, from 2,4- and/or 2,6-tolylene diisocyanate (TDI, R²=C₆H₃—CH₃), 4,4′-methylenedi(phenyl isocyanate) (MDI, R²=C₆H₄—CH₂—C₆H₄) or hexamethylene diisocyanate [HMDI, R²=(CH₂)₆].

The plastics mentioned may be used alone as materials for the elevations in the upper punch which mold the reinforcement depressions, but may also be provided with coatings or laminations of metals or other substances. In the context of the present invention, it has been found to be particularly appropriate to use glass fiber reinforced plastics as material for the elevations in the upper punch which mold the reinforcement depressions. Glass fiber reinforced plastics (GRPs) are composite materials comprising a combination of a polymer matrix and reinforcing glass fibers. The glass materials used for fiber reinforcement in the GRPs are in the form of fibers, yarns, rovings (glass silk strands), nonwovens, wovens or mats. Suitable polymeric matrix systems for GRPs include both thermosets (such as epoxy resins, unsaturated polyester resins, phenolic resins and furan resins, for example) and thermoplastics (such as polyamides, polycarbonates, polyacetals, polyphenylene oxides and sulfides, polypropylenes and styrene copolymers, for example). The weight ratio between reinforcing material and polymer matrix is usually in the range of 10:90-65:35, the strength properties of the GRPs generally increasing up to a reinforcement content of approximately 40% by weight.

The GRPs are produced predominantly in compression processes; further important manufacturing processes are hand layup, spray layup, continuous impregnation, filament winding and centrifugal processes. In many cases the process starts from what are known as prepregs, glass fiber materials preimpregnated with resins, which are cured using heat and pressure. Relative to the unreinforced matrix polymers, the GRPs feature increased tensile, flexural and compressive strength, impact toughness, dimensional stability, and stability with respect to the effect of heat, acids, salts, gases or solvents. In the context of the present invention, glass fiber reinforced polytetrafluoroethylene and glass fiber reinforced polyamides have been found to be particularly appropriate materials for the elevations in the upper punch which mold the reinforcement depressions.

Suitable materials for the base surface here too are plastics which are softer than the plastics used for the construction of the elevations in the upper punch which mold the reinforcement depressions. It will be appreciated that these plastics may stem from the same or else from different groups, provided that the requirement on the hardness or softness is fulfilled. The preferably adhesion-preventing or at least adhesion-reducing action of the flexible material for the formation of the base surface has been described above. In experiments, it has been found that very good results were achievable with the polyurethane material Vulkollan or the PVC material Mipolam. Over use times of several thousand pressings, no adhesions whatsoever on the base surface material were found.

It is possible with the process according to the invention to produce tablets of a wide variety of different compositions, the process according to the invention minimizing, in particular, the problems in the production and use of cleaning composition tablets for machine dishwashing. These cleaning composition tablets comprise typically only minor amounts of surfactants. Washing composition tablets are typically produced by blending surfactant granules with formulation components and subsequently pressing this particulate premixture. Preferred variants of the process according to the invention are therefore characterized in that the particulate premixture comprises surfactant-containing granule(s) and has a bulk density of at least 500 g/l, preferably at least 600 g/l and, in particular, at least 700 g/l.

Processes preferred in context of the present invention therefore include the pressing of a particular premixture of at least one surfactant-containing granule and at least one admixed pulverulent component. The surfactant-containing granules can be produced by customary industrial granulation processes such as compaction, extrusion, mixer granulation, pelletization or fluidized bed granulation.

In preferred process variants, the surfactant-containing granule fulfills certain particle size criteria. Preference is thus given to processes according to the invention in which the surfactant-containing granule has particle sizes between 100 and 2,000 μm, preferably between 200 and 1,800 μm, more preferably between 400 and 1600 μm and, in particular, between 600 and 1,400 μm.

In addition to the active substances (anionic and/or nonionic and/or cationic and/or amphoteric surfactants), the surfactant granules preferably also comprise carriers which more preferably stem from the group of the builders. Particularly advantageous processes are characterized in that the surfactant-containing granule comprises anionic and/or nonionic surfactants and builders, and has total surfactant contents of at least 10% by weight, preferably at least 15% by weight and, in particular, at least 20% by weight.

These interface-active substances stem from the group of the anionic, nonionic, zwitterionic and cationic surfactants, anionic surfactants being distinctly preferred for economic reasons and owing to their performance spectrum.

The anionic surfactants used are, for example, those of the sulfonate and sulfate type. Useful surfactants of the sulfonate type are preferably C₉₋₁₃-alkylbenzenesulfonates, olefinsulfonates, i.e. mixtures of alkene- and hydroxyalkanesulfonates, and disulfonates, as are obtained, for example, from C₁₂₋₁₈-monoolefins with terminal or internal double bond by sulfonation with gaseous sulfur trioxide and subsequent alkaline or acidic hydrolysis of the sulfonation products. Also suitable are alkanesulfonates which are obtained from C₁₂₋₁₈-alkanes, for example, by sulfochlorination or sulfoxidation with subsequent hydrolysis or neutralization. The esters of α-sulfo fatty acids (ester sulfonates), for example, the α-sulfonated methyl esters of hydrogenated coconut, palm kernel or tallow fatty acids, are also likewise suitable.

Further suitable anionic surfactants are sulfated fatty acid glycerol esters. Fatty acid glycerol esters refer to the mono-, di- and triesters, and mixtures thereof, as are obtained in the preparation by esterification of a monoglycerol with from 1 to 3 mol of fatty acid or in the transesterification of triglycerides with from 0.3 to 2 mol of glycerol. Preferred sulfated fatty acid glycerol esters are the sulfation products of saturated fatty acids having from 6 to 22 carbon atoms, for example, of caproic acid, caprylic acid, capric acid, myristic acid, lauric acid, palmitic acid, stearic acid or behenic acid.

Preferred alk(en)yl sulfates are the alkali metal and, in particular, the sodium salts of the sulfuric monoesters of C₁₂-C₁₈ fatty alcohols, for example, of coconut fatty alcohol, tallow fatty alcohol, lauryl, myristyl, cetyl or stearyl alcohol, or of C₁₀-C₂₀ oxo alcohols and those monoesters of secondary alcohols of these chain lengths. Also preferred are alk(en)yl sulfates of the chain length mentioned which contain a synthetic straight-chain alkyl radical prepared on a petrochemical basis and which have analogous degradation behavior to the equivalent compounds based on fatty chemical raw materials. From the washing point of view, preference is given to the C₁₂-C₁₆-alkyl sulfates and C₁₂-C₁₅-alkyl sulfates, and C₁₄-C₁₅-alkyl sulfates. 2,3-Alkyl sulfates, which are prepared, for example, as described in U.S. Pat. No. 3,234,258 or U.S. Pat. No. 5,075,041 and can be obtained as commercial products from the Shell Oil Company under the name DAN®, are also suitable anionic surfactants.

Also suitable are the sulfuric monoesters of the straight-chain or branched C₇₋₂₁-alcohols ethoxylated with 1 to 6 mol of ethylene oxide, such as 2-methyl-branched C₉₋₁₁-alcohols with on average 3.5 mol of ethylene oxide (EO) or C₁₂₋₁₈-fatty alcohols with from 1 to 4 EO. Owing to their high tendency to foam, they are used in cleaning compositions only in relatively small amounts, for example, amounts of from 1 to 5% by weight.

Further suitable anionic surfactants are also the salts of alkylsulfosuccinic acid, which are also referred to as sulfosuccinates or as sulfosuccinic esters and are the monoesters and/or diesters of sulfosuccinic acid with alcohols, preferably fatty alcohols and, in particular, ethoxylated fatty alcohols. Preferred sulfosuccinates contain C₈₋₁₈ fatty alcohol radicals or mixtures thereof. Especially preferred sulfosuccinates contain a fatty alcohol radical which is derived from ethoxylated fatty alcohols which, considered alone, constitute nonionic surfactants (for description see below). In this context, particular preference is again given to sulfosuccinates whose fatty alcohol radicals are derived from ethoxylated fatty alcohols with a narrowed homolog distribution. It is also equally possible to use alk(en)ylsuccinic acid having preferably from 8 to 18 carbon atoms in the alk(en)yl chain or salts thereof.

Useful further anionic surfactants are, in particular, soaps. Suitable soaps are saturated fatty acid soaps, such as the salts of lauric acid, myristic acid, palmitic acid, stearic acid, hydrogenated erucic acid and behenic acid, and soap mixtures derived, in particular, from natural fatty acids, for example, coconut, palm kernel or tallow fatty acids.

The anionic surfactants including the soaps may be present in the form of their sodium, potassium or ammonium salts, and also in the form of soluble salts of organic bases, such as mono-, di- or triethanolamine. The anionic surfactants are preferably present in the form of their sodium or potassium salts, in particular, in the form of the sodium salts.

In the context of the present invention, preference is given to surfactant granules which contain from 5 to 50% by weight, preferably from 7.5 to 40% by weight and, in particular, from 10 to 30% by weight of anionic surfactant(s), based in each case on the granule.

In the selection of the anionic surfactants which are used, there are no boundary conditions to be observed which obstruct the freedom to formulate. However, preferred surfactant granules have a content of soap which exceeds 0.2% by weight based on the washing and cleaning composition tablet produced in step d). Anionic surfactants to be used with preference are the alkylbenzenesulfonates and fatty alcohol sulfates, preferred washing and cleaning composition tablets containing from 2 to 20% by weight, preferably from 2.5 to 15% by weight and, in particular, from 5 to 10% by weight of fatty alcohol sulfate(s), based in each case on the weight of the washing and cleaning composition tablets.

When the inventive tablets are used as machine dishwasher detergents, they preferably comprise only minor amounts of anionic surfactants, but rather mainly nonionic surfactants.

The nonionic surfactants used are preferably alkoxylated, advantageously ethoxylated, in particular, primary alcohols having preferably from 8 to 18 carbon atoms and on average from 1 to 12 mol of ethylene oxide (EO) per mole of alcohol in which the alcohol radical may be linear or preferably 2-methyl-branched, or may contain a mixture of linear and methyl-branched radicals, as are typically present in oxo alcohol radicals. However, especially preferred alcohol ethoxylates have linear radicals of alcohols of natural origin having from 12 to 18 carbon atoms, for example, of coconut, palm, tallow fat or oleyl alcohol, and on average from 2 to 8 EO per mole of alcohol. The preferred ethoxylated alcohols include, for example, C₁₂₋₁₄-alcohols having 3 EO or 4 EO, C₉₋₁₁-alcohol having 7 EO, C₁₃₋₁₅-alcohols having 3 EO, 5 EO, 7 EO or 8 EO, C₁₂₋₁₈-alcohols having 3 EO, 5 EO or 7 EO and mixtures thereof, such as mixtures of C₁₂₋₁₄-alcohol having 3 EO and C₁₂₋₁₈-alcohol having 5 EO. The degrees of ethoxylation specified are statistical average values which may be an integer or a fraction for a specific product. Preferred alcohol ethoxylates have a narrowed homolog distribution (narrow range ethoxylates, NRE). In addition to these nonionic surfactants, it is also possible to use fatty alcohols having more than 12 EO. Examples thereof are tallow fatty alcohol having 14 EO, 25 EO, 30 EO or 40 EO.

In addition, further nonionic surfactants which may be used are also alkyl glycosides of the general formula RO(G)ₓ in which R is a primary straight-chain or methyl-branched, in particular, 2-methyl-branched, aliphatic radical having from 8 to 22, preferably from 12 to 18, carbon atoms and G is the symbol which is a glycose unit having 5 or 6 carbon atoms, preferably glucose. The degree of oligomerization x, which specifies the distribution of monoglycosides and oligoglycosides, is any number between 1 and 10; x is preferably from 1.2 to 1.4.

A further class of nonionic surfactants used with preference, which are used either as the sole nonionic surfactant or in combination with other nonionic surfactants, are alkoxylated, preferably ethoxylated or ethoxylated and propoxylated, fatty acid alkyl esters, preferably having from 1 to 4 carbon atoms in the alkyl chain.

Nonionic surfactants of the amine oxide type, for example, N-cocoalkyl-N,N-dimethylamine oxide and N-tallowalkyl-N,N-dihydroxyethylamine oxide, and of the fatty acid alkanolamide type may also be suitable. The amount of these nonionic surfactants is preferably not more than that of the ethoxylated fatty alcohols, in particular, not more than half thereof.

Further suitable surfactants are polyhydroxy fatty acid amides of the formula 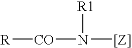
in which RCO is an aliphatic acyl radical having from 6 to 22 carbon atoms, R¹ is hydrogen, an alkyl or hydroxyalkyl radical having from 1 to 4 carbon atoms and [Z] is a linear or branched polyhydroxyalkyl radical having from 3 to 10 carbon atoms and from 3 to 10 hydroxyl groups. The polyhydroxy fatty acid amides are known substances which can typically be obtained by reductively aminating a reducing sugar with ammonia, an alkylamine or an alkanolamine, and subsequently acylating with a fatty acid, a fatty acid alkyl ester or a fatty acid chloride.

The group of polyhydroxy fatty acid amides also includes compounds of the formula 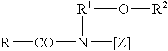
in which R is a linear or branched alkyl or alkenyl radical having from 7 to 12 carbon atoms, R¹ is a linear, branched or cyclic alkyl radical or an aryl radical having from 2 to 8 carbon atoms and R² is a linear, branched or cyclic alkyl radical or an aryl radical or an oxyalkyl radical having from 1 to 8 carbon atoms, preference being given to C₁₋₄-alkyl or phenyl radicals, and [Z] is a linear polyhydroxyalkyl radical whose alkyl chain is substituted by at least two hydroxyl groups, or alkoxylated, preferably ethoxylated or propoxylated, derivatives of this radical.

[Z] is preferably obtained by reductive amination of a reduced sugar, for example, glucose, fructose, maltose, lactose, galactose, mannose or xylose. The N-alkoxy- or N-aryloxy-substituted compounds can be converted to the desired polyhydroxy fatty acid amides by reaction with fatty acid methyl esters in the presence of an alkoxide as catalyst.

The surfactants used with preference are low-foaming nonionic surfactants. With particular preference, the inventive cleaning compositions for machine dishwashing comprise nonionic surfactants, in particular, nonionic surfactants from the group of the alkoxylated alcohols. The nonionic surfactants used are preferably alkoxylated, advantageously ethoxylated, in particular, primary alcohols having preferably from 8 to 18 carbon atoms and on average from 1 to 12 mol of ethylene oxide (EO) per mole of alcohol in which the alcohol radical may be linear or preferably 2-methyl-branched, or may contain a mixture of linear and methyl-branched radicals, as are typically present in oxo alcohol radicals. However, especially preferred are alcohol ethoxylates having linear radicals of alcohols of natural origin having from 12 to 18 carbon atoms, for example, of coconut, palm, tallow fat or oleyl alcohol, and on average from 2 to 8 EO per mole of alcohol. The preferred ethoxylated alcohols include, for example, C₁₂₋₁₄-alcohols having 3 EO or 4 EO, C₉₋₁₁-alcohol having 7 EO, C₁₃₋₁₅-alcohols having 3 EO, 5 EO, 7 EO or 8 EO C₁₂₋₁₈-alcohols having 3 EO, 5 EO or 7 EO and mixtures thereof, such as mixtures of C₁₂₋₁₄-alcohol having 3 EO and C₁₂₋₁₈-alcohol having 5 EO. The degrees of ethoxylation specified are statistical average values which may be an integer or a fraction for a specific product. Preferred alcohol ethoxylates have a narrowed homolog distribution (narrow range ethoxylates, NRE). In addition to these nonionic surfactants, it is also possible to use fatty alcohols having more than 12 EO. Examples thereof are tallow fatty alcohol having 14 EO, 25 EO, 30 EO or 40 EO.

Special preference is given to inventive compositions which comprise a nonionic surfactant which has a melting point above room temperature. Accordingly, preferred dishwasher detergents are characterized in that they comprise nonionic surfactant(s) having a melting point above 20° C., preferably above 25° C., more preferably between 25 and 60° C. and in particular, between 26.6 and 43.3° C.

Suitable nonionic surfactants which have melting or softening points in the temperature range specified are, for example, low-foaming nonionic surfactants which may be solid or highly viscous at room temperature. When nonionic surfactants which have a high viscosity at room temperature are used, they preferably have a viscosity above 20 Pas, preferably above 35 Pas and in particular, above 40 Pas. Nonionic surfactants which have a waxlike consistency at room temperature are also preferred.

Nonionic surfactants which are solid at room temperature and are to be used with preference stem from the groups of alkoxylated nonionic surfactants, in particular, the ethoxylated primary alcohols and mixtures of these surfactants with structurally more complex surfactants, such as polyoxypropylene/polyoxyethylene/polyoxypropylene (PO/EO/PO) surfactants. Such (PO/EO/PO) nonionic surfactants are additionally notable for good foam control.

In a preferred embodiment of the present invention, the nonionic surfactant with a melting point above room temperature is an ethoxylated nonionic surfactant which has resulted from the reaction of a monohydroxyalkanol or alkylphenol having from 6 to 20 carbon atoms with preferably at least 12 mol, more preferably at least 15 mol, in particular, at least 20 mol, of ethylene oxide per mole of alcohol or alkylphenol.

A nonionic surfactant which is solid at room temperature and is to be used with particular preference is obtained from a straight-chain fatty alcohol having from 16 to 20 carbon atoms (C₁₆₋₂₀-alcohol), preferably a C₁₈-alcohol, and at least 12 mol, preferably at least 15 mol and in particular, at least 20 mol, of ethylene oxide. Of these, the “narrow range ethoxylates” (see above) are particularly preferred.

Accordingly, particularly preferred inventive dishwasher detergents comprise ethoxylated nonionic surfactant(s) which has/have been obtained from C₆₋₂₀-monohydroxyalkanols or C₆₋₂₀-alkylphenols or C₁₆₋₂₀-fatty alcohols and more than 12 mol, preferably more than 15 mol and in particular, more than 20 mol of ethylene oxide per mole of alcohol.

The room temperature solid nonionic surfactant preferably additionally has propylene oxide units in the molecule. Preferably, such PO units make up up to 25% by weight, more preferably up to 20% by weight and in particular, up to 15% by weight, of the total molar mass of the nonionic surfactant. Particularly preferred nonionic surfactants are ethoxylated monohydroxyalkanols or alkylphenols which additionally have polyoxyethylene-polyoxypropylene block copolymer units. The alcohol or alkylphenol moiety of such nonionic surfactant molecules preferably makes up more than 30% by weight, more preferably more than 50% by weight and in particular, more than 70% by weight, of the total molar mass of such nonionic surfactants. Preferred dishwasher detergents are characterized in that they comprise ethoxylated and propoxylated nonionic surfactants in which the propylene oxide units in the molecule make up up to 25% by weight, preferably up to 20% by weight and in particular, up to 15% by weight, of the total molar mass of the nonionic surfactant.

Further nonionic surfactants which have melting points above room temperature and are to be used with particular preference contain from 40 to 70% of a polyoxypropylene/polyoxyethylene/polyoxypropylene block polymer blend which contains 75% by weight of an inverse block copolymer of polyoxyethylene and polyoxypropylene having 17 mol of ethylene oxide and 44 mol of propylene oxide, and 25% by weight of a block copolymer of polyoxyethylene and polyoxypropylene initiated with trimethylolpropane and containing 24 mol of ethylene oxide and 99 mol of propylene oxide per mole of trimethylolpropane.

Nonionic surfactants which can be used with particular preference are obtainable, for example, under the name Poly Tergent® SLF-18 from Olin Chemicals.

A more preferred inventive machine dishwasher detergent comprises nonionic surfactants of the formula (VI)
R¹O[CH₂CH(CH₃)O]ₓ[CH₂CH₂O]_{y}[CH₂CH(OH)R²] (VI)
in which R¹ is a linear or branched aliphatic hydrocarbon radical having from 4 to 18 carbon atoms or mixtures thereof, R² is a linear or branched hydrocarbon radical having from 2 to 26 carbon atoms or mixtures thereof, and x is a value between 0.5 and 1.5, and y is a value of at least 15.

Further nonionic surfactants which can be used with preference are the terminally capped poly(oxyalkylated) nonionic surfactants of the formula
R¹O[CH₂CH(R³)O]ₓ[CH₂]ₖCH(OH)[CH₂]ⱼOR²
in which R¹ and R² are linear or branched, saturated or unsaturated, aliphatic or aromatic hydrocarbon radicals having from 1 to 30 carbon atoms, R³ is H or a methyl, ethyl, n-propyl, isopropyl, n-butyl, 2-butyl or 2-methyl-2-butyl radical, x is a value between 1 and 30, k and j are values between 1 and 12, preferably between 1 and 5. When the value x is ≧2, each R³ in the above formula may be different. R¹ and R² are preferably linear or branched, saturated or unsaturated, aliphatic or aromatic hydrocarbon radicals having from 6 to 22 carbon atoms, particular preference being given to radicals having from 8 to 18 carbon atoms. For the R³ radical, particular preference is given to H, —CH₃ or —CH₂CH₃. Particularly preferred values for x are in the range from 1 to 20, in particular, from 6 to 15.

As described above, each R³ in the above formula may be different if x is ≧2. This allows the alkylene oxide unit in the square brackets to be varied. When x is, for example, 3, the R³ radical may be selected so as to form ethylene oxide (R³=H) or propylene oxide (R³=CH₃) units which can be joined together in any sequence, for example, (EO)(PO)(EO), (EO)(EO)(PO), (EO)(EO)(EO), (PO)(EO)(PO), (PO)(PO)(EO) and (PO)(PO)(PO). The value 3 for x has been selected here by way of example and it is entirely possible for it to be larger, the scope of variation increasing with increasing x values and embracing, for example, a large number of (EO) groups combined with a small number of (PO) groups, or vice versa.

Especially preferred terminally capped poly(oxyalkylated) alcohols of the above formula have values of k=1 and j=1, so that the above formula is simplified to
R¹O[CH₂CH(R³)O]ₓCH₂CH(OH)CH₂OR².
In the latter formula, R¹, R² and R³ are each as defined above and x is a number from 1 to 30, preferably from 1 to 20 and in particular, from 6 to 18. Particular preference is given to surfactants in which the R¹ and R² radicals have from 9 to 14 carbon atoms, R³ is H and x assumes values of from 6 to 15.

If the latter statements are summarized, preference is given to inventive dishwasher detergents which comprise the terminally capped poly(oxyalkylated) nonionic surfactants of the formula
R¹O[CH₂CH(R³)O]ₓ[CH₂]ₖCH(OH)[CH₂]ⱼOR²
in which R¹ and R² are linear or branched, saturated or unsaturated, aliphatic or aromatic hydrocarbon radicals having from 1 to 30 carbon atoms, R³ is H or a methyl, ethyl, n-propyl, isopropyl, n-butyl, 2-butyl or 2-methyl-2-butyl radical, x is a value between 1 and 30, k and j are values between 1 and 12, preferably between 1 and 5, particular preference being given to surfactants of the
R¹O[CH₂CH(R³)O]ₓCH₂CH(OH)CH₂OR²
type in which x is a number from 1 to 30, preferably from 1 to 20 and in particular, from 6 to 18.

Particularly preferred nonionic surfactants in the context of the present invention have been found to be low-foaming nonionic surfactants which have alternating ethylene oxide and alkylene oxide units. Among these, preference is given in turn to surfactants having EO-AO-EO-AO blocks, and in each case from one to ten EO and/or AO groups are bonded to one another before a block of the other groups in each case follows. Preference is given here to inventive machine dishwasher detergents which comprise, as nonionic surfactant(s), surfactants of the general formula VII
R¹—O—(CH₂—CH₂—O)_{w}—(CH₂—CH(R²)—O)ₓ—(CH₂—CH₂—O)_{y}—(CH₂—CH(R³)—O)_{z}—H (VII)
in which R¹ is a straight-chain or branched, saturated or mono- or polyunsaturated C₆₋₂₄-alkyl or -alkenyl radical; each R² or R³ group is independently selected from —CH₃; —CH₂CH₃, —CH₂CH₂—CH₃, CH(CH₃)₂ and the indices w, x, y, z are each independently integers from 1 to 6.

The preferred nonionic surfactants of the formula VII can be prepared by known methods from the corresponding alcohols R¹—OH and ethylene oxide or alkylene oxide. The R¹ radical in the above formula III may vary depending on the origin of the alcohol. When native sources are utilized, the R¹ radical has an even number of carbon atoms and is generally unbranched, and preference is given to the linear radicals of alcohols of native origin having from 12 to 18 carbon atoms, for example, from coconut, palm, tallow fat or oleyl alcohol. Alcohols obtainable from synthetic sources are, for example, the Guerbet alcohols or 2-methyl-branched or linear and methyl-branched radicals in a mixture, as are typically present in oxo alcohol radicals. Irrespective of the type of the alcohol used to prepare the nonionic surfactants present in accordance with the invention in the compositions, preference is given to inventive machine dishwasher detergents in which R¹ in formula VII is an alkyl radical having from 6 to 24, preferably from 8 to 20, more preferably from 9 to 15 and in particular, from 9 to 11 carbon atoms.

The alkylene oxide unit which is present in the preferred nonionic surfactants in alternation to the ethylene oxide unit is, as well as propylene oxide, especially butylene oxide. However, further alkylene oxides in which R² and R³ are each independently selected from —CH₂CH₂—CH₃ and CH(CH₃)₂ are also suitable. Preferred machine dishwasher detergents are characterized in that R² and R³ are each a —CH₃ radical, w and x are each independently 3 or 4, and y and z are each independently 1 or 2.

In summary, for use in the inventive compositions, preference is given in particular, to nonionic surfactants which have a C₉₋₁₅-alkyl radical having from 1 to 4 ethylene oxide units, followed by from 1 to 4 propylene oxide units, followed by from 1 to 4 ethylene oxide units, followed by from 1 to 4 propylene oxide units. In aqueous solution, these surfactants have the required low viscosity and can be used with particular preference in accordance with the invention.

Further nonionic surfactants usable with preference are the terminally capped poly(oxyalkylated) nonionic surfactants of the formula (VIII)
R¹O[CH₂CH(R³)O]ₓR² (VIII)
in which R¹ is linear or branched, saturated or unsaturated, aliphatic or aromatic hydrocarbon radicals having from 1 to 30 carbon atoms, R² is linear or branched, saturated or unsaturated, aliphatic or aromatic hydrocarbon radicals having from 1 to 30 carbon atoms and preferably having between 1 and 5 hydroxyl groups and are preferably further functionalized with an ether group, R³ is H or a methyl, ethyl, n-propyl, isopropyl, n-butyl, 2-butyl or 2-methyl-2-butyl radical, x is between 1 and 40.

In particularly preferred nonionic surfactants of the above formula (VIII), R³ is H. In the resulting terminally capped poly(oxyalkylated) nonionic surfactants of the formula (IX)
R¹O[CH₂CH₂O]ₓR² (IX),
preference is given in particular, to those nonionic surfactants in which R¹ is linear or branched, saturated or unsaturated, aliphatic or aromatic hydrocarbon radicals having from 1 to 30 carbon atoms, preferably having from 4 to 20 carbon atoms, R² is linear or branched, saturated or unsaturated, aliphatic or aromatic hydrocarbon radicals having from 1 to 30 carbon atoms and which preferably have between 1 and 5 hydroxyl groups, and x is values between 1 and 40.

Preference is given in particular, to those terminally capped poly(oxyalkylated) nonionic surfactants which, according to the formula (X)
R¹O[CH₂CH₂O]ₓCH₂CH(OH)R² (X),
have not only an R¹ radical which is linear or branched, saturated or unsaturated, aliphatic or aromatic hydrocarbon radicals having from 1 to 30 carbon atoms, preferably having from 4 to 20 carbon atoms, but also a linear or branched, saturated or unsaturated, aliphatic or aromatic hydrocarbon radical having from 1 to 30 carbon atoms R² which is adjacent to a monohydroxylated intermediate group —CH₂CH(OH)—. In this formula, x is values between 1 and 40. Such terminally capped poly(oxyalkylated) nonionic surfactants can be obtained, for example, by reacting a terminal epoxide of the formula R²CH(O)CH₂ with an ethoxylated alcohol of the formula R¹O[CH₂CH₂O]ₓ₋₁CH₂CH₂OH.

The specified carbon chain lengths and degrees of ethoxylation or degrees of alkoxylation of the aforementioned nonionic surfactants constitute statistical averages which may be a whole number or a fraction for a specific product. As a consequence of the preparation process, commercial products of the formulas specified do not usually consist of one individual representative, but rather of mixtures, as a result of which average values and consequently fractions can arise both for the carbon chain lengths and for the degrees of ethoxylation or degrees of alkoxylation.

A further class of nonionic surfactants which may be used advantageously is that of the alkyl polyglycosides (APG). Usable alkyl polyglycosides satisfy the general formula RO(G)_{z} in which R is a linear or branched, in particular, 2-methyl-branched, saturated or unsaturated aliphatic radical having from 8 to 22, preferably from 12 to 18, carbon atoms and G is the symbol which represents a glycose unit having 5 or 6 carbon atoms, preferably glucose. The degree of glycosylation z is between 1.0 and 4.0, preferably between 1.0 and 2.0 and in particular, between 1.1 and 1.4.

Preference is given to using linear alkyl polyglucosides, i.e. alkyl polyglycosides in which the polyglycosyl radical is a glucose radical and the alkyl radical is an n-alkyl radical.

The surfactant granules may preferably comprise alkyl polyglycosides, preference being given to contents of APG above 0.2% by weight based on the overall tablet. Particularly preferred washing and cleaning composition tablets comprise APG in amounts of from 0.2 to 10% by weight, preferably from 0.2 to 5% by weight and in particular, from 0.5 to 3% by weight.

Nonionic surfactants of the amine oxide type, for example, N-cocoalkyl-N,N-dimethylamine oxide and N-tallowalkyl-N,N-dihydroxyethylamine oxide, and the fatty acid alkanolamides may also be suitable. The amount of these nonionic surfactants is preferably not more than that of the ethoxylated fatty alcohols, especially not more than half thereof.

Irrespective of whether anionic or nonionic surfactants or mixtures of these surfactant classes and, if appropriate, amphoteric or cationic surfactants are used in the surfactant granule, preference is given to processes according to the invention in which the surfactant content of the surfactant-containing granule is from 5 to 60% by weight, preferably from 10 to 50% by weight and in particular, from 15 to 40% by weight, based in each case on the surfactant granule.

The surfactant granule may be used in varying amounts in the washing and cleaning composition tablets. Preference is given to processes according to the invention in which the fraction of the surfactant-containing granule in the washing and cleaning composition tablets is from 40 to 95% by weight, preferably from 45 to 85% by weight and in particular, from 55 to 75% by weight, based in each case on the weight of the washing and cleaning composition tablets. As already mentioned above, cleaning composition tablets for machine dishwashing typically comprise only small amounts of surfactants, so that the above statements do not apply to this class of cleaning composition tablets.

In addition to the washing-active substances, builders are the most important ingredients of washing and cleaning compositions. In the surfactant granules or where no surfactant granules are used, it is also possible for all builders used customarily in washing and cleaning compositions to be present as a constituent of the premixture, i.e. especially zeolites, silicates, carbonates, organic builders and, where no ecological objections to their use exist, also the phosphates.

Suitable crystalline, sheet-type sodium silicates have the general formula NaMSiₓO₂ₓ₊₁≠H₂O where M is sodium or hydrogen, x is a number from 1.9 to 4, y is a number from 0 to 20, and preferred values for x are 2, 3 or 4. Preferred crystalline sheet silicates of the formula specified are those in which M is sodium and x assumes the values of 2 or 3. In particular, preference is given to both β- and also δ-sodium disilicates Na₂Si₂O₅≠yH₂O.

It is also possible to use amorphous sodium silicates having an Na₂O:SiO₂ modulus of from 1:2 to 1:3.3, preferably from 1:2 to 1:2.8 and in particular, from 1:2 to 1:2.6, which have retarded dissolution and secondary washing properties. The retardation of dissolution relative to conventional amorphous sodium silicates may have been brought about in a variety of ways, for example, by surface treatment, compounding, compacting or by overdrying. In the context of this invention, the term morphous also includes -ray-amorphous This means that, in X-ray diffraction experiments, the silicates do not afford any sharp X-ray reflections typical of crystalline substances, but rather yield at best one or more maxima of the scattered X-radiation, which have a width of several degree units of the diffraction angle. However, it may quite possibly lead to even particularly good builder properties if the silicate particles in electron diffraction experiments yield vague or even sharp diffraction maxima. This is to be interpreted such that the products have microcrystalline regions with a size of from 10 to several hundred nm, preference being given to values up to a maximum of 50 nm and in particular, up to a maximum of 20 nm. Such X-ray-amorphous silicates likewise have retarded dissolution compared with conventional waterglasses. Special preference is given to compacted amorphous silicates, compounded amorphous silicates and overdried X-ray-amorphous silicates.

Washing and cleaning compositions preferred in the context of the present invention are characterized in that these silicate(s), preferably alkali metal silicates, more preferably crystalline or amorphous alkali metal disilicates, are present in amounts of from 10 to 60% by weight, preferably from 15 to 50% by weight and in particular, from 20 to 40% by weight, based in each case on the weight of the washing or cleaning composition.

When the inventive compositions are used as machine dishwasher detergents, these compositions preferably comprise at least one crystalline sheet-type silicate of the general formula NaMSiₓO₂ₓ₊₁x+·yH₂O where M is sodium or hydrogen, x is a number from 1.9 to 22, preferably from 1.9 to 4, and y is a number from 0 to 33. The crystalline sheet-type silicates of the formula (I) are sold, for example, by Clariant GmbH (Germany) under the trade name Na-SKS, for example, Na-SKS-1 (Na₂Si₂₂O₄₅·xH₂O, kenyaite), Na-SKS-2 (Na₂Si₁₄O₂₉·xH₂O, magadiite), Na-SKS-3 (Na₂Si₈O₁₇·xH₂O) or Na-SKS-4 (Na₂Si₄O₉·xH₂O, makatite).

Particularly suitable for the purposes of the present invention are compositions which comprise crystalline sheet silicates of the formula (I) in which x is 2. Among these, suitable in particular, are Na-SKS-5 (α-Na₂Si₂O₅), Na-SKS-7 (β-Na₂Si₂O₅, natrosilite), Na-SKS-9 (NaHSi₂O₅·H₂O), Na-SKS-10 (NaHSi₂O₅·3H₂O, kanemite), Na-SKS-11 (t-Na₂Si₂O₅) and Na-SKS-13 (NaHSi₂O₅), but in particular, Na-SKS-6 (δ-Na₂Si₂O₅). An overview of crystalline sheet silicates can be found, for example, in the article published in Seifen-Öle-Fette-Wachse, 116, No. 20/1990 on pages 805-808.

In the context of the present application, preferred machine dishwasher detergents or machine dishwasher assistants comprise a proportion by weight of the crystalline sheet-type silicate of the formula (I) of from 0.1 to 20% by weight, preferably from 0.2 to 15% by weight and in particular, from 0.4 to 10% by weight, based in each case on the total weight of these compositions. Particular preference is given especially to those machine dishwasher detergents which have a total silicate content below 7% by weight, preferably below 6% by weight, preferentially below 5% by weight, more preferably below 4% by weight, even more preferably below 3% by weight and in particular, below 2.5% by weight, this silicate, based on the total weight of the silicate present, being silicate of the general formula NaMSiₓO₂ₓ₊₁·yH₂O preferably to an extent of at least 70% by weight, preferentially to an extent of at least 80% by weight and in particular, to an extent of at least 90% by weight.

The finely crystalline, synthetic, bound water-containing zeolite used is preferably zeolite A and/or P. The zeolite P is more preferably Zeolite MAP® (commercial product from Crosfield). Also suitable, however, are zeolite X, and mixtures of A, X and/or P. Also commercially available and usable with preference in accordance with the present invention is, for example, a cocrystal of zeolite X and zeolite A (approximately 80% by weight of zeolite X), which is sold by CONDEA Augusta S.p.A. under the trade name VEGOBOND AX® and can be described by the formula
nNa₂O ·(1-n)K₂O·Al₂O₃·(2-2.5)SiO₂ ·(3.5-5.5) H₂O.

The zeolite may be used either as a builder in a granular compound or in a kind of “powdering” of the entire mixture to be compacted, and both ways of incorporating the zeolite into the premixture are typically utilized. Suitable zeolites have an average particle size of less than 10 μm (volume distribution; measurement method: Coulter Counter) and preferably contain from 18 to 22% by weight, in particular, from 20 to 22% by weight, of bound water.

If desired, it is possible to incorporate further zeolite over and above the amount of zeolite of the P and/or X type introduced by the surfactant granule into the premixture by adding zeolite as a formulation component. The finely crystalline, synthetic zeolite comprising bound water used is preferably a zeolite of the A, P, X or Y type. However, also suitable is zeolite X and mixtures of zeolite A, X and/or P. Suitable zeolites have a mean particle size of less than 10 μm (volume distribution; measurement method: Coulter counter) and contain preferably from 18 to 22% by weight, in particular, from 20 to 22% by weight of bound water.

It is of course also possible to use the commonly known phosphates as builder substances, as long as such a use is not to be avoided for ecological reasons. This is especially true for the use of inventive compositions as machine dishwasher detergents, which is particularly preferred in the context of the present application. Among the multitude of commercially available phosphates, the alkali metal phosphates, with particular preference for pentasodium triphosphate or pentapotassium triphosphate (sodium tripolyphosphate or potassium tripolyphosphate), have the greatest significance in the washing and cleaning products industry.

Alkali metal phosphates is the collective term for the alkali metal (especially sodium and potassium) salts of the various phosphoric acids, for which a distinction may be drawn between metaphosphoric acids (HPO₃)ₙ and orthophosphoric acid H₃PO₄, in addition to higher molecular weight representatives. The phosphates combine a number of advantages: they act as alkali carriers, prevent limescale deposits on machine components and lime encrustations in fabrics, and additionally contribute to the cleaning performance.

Sodium dihydrogenphosphate, NaH₂PO₄, exists as the dihydrate (density 1.91 gcm⁻³, melting point 60°) and as the monohydrate (density 2.04 gcm⁻³). Both salts are white powders which are very readily soluble in water and which lose the water of crystallization upon heating and are converted at 200° C. to the weakly acidic diphosphate (disodium hydrogendiphosphate, Na₂H₂P₂O₇), and at higher temperature to sodium trimetaphosphate (Na₃P₃O₉) and Maddrell salt (see below). NaH₂PO₄ reacts acidically; it is formed when phosphoric acid is adjusted to a pH of 4.5 using sodium hydroxide solution and the slurry is sprayed. Potassium dihydrogenphosphate (primary or monobasic potassium phosphate, potassium biphosphate, KDP), KH₂PO₄, is a white salt of density of 2.33 gcm⁻³, has a melting point of 253° [decomposition with formation of potassium polyphosphate (KPO₃)ₓ] and is readily soluble in water.

Disodium hydrogenphosphate (secondary sodium phosphate), Na₂HPO₄, is a colorless crystalline salt which is very readily soluble in water. It exists in anhydrous form and with 2 mol of water (density 2.066 gcm⁻³, loss of water at 95°), 7 mol of water (density 1.68 gcm⁻³, melting point 48° with loss of 5H₂O) and 12 mol of water (density 1.52 gcm⁻³, melting point 35° with loss of 5H₂O), becomes anhydrous at 100° and, when heated more strongly, is converted to the diphosphate Na₄P₂O₇. Disodium hydrogenphosphate is prepared by neutralizing phosphoric acid with sodium carbonate solution using phenolphthalein as an indicator. Dipotassium hydrogenphosphate (secondary or dibasic potassium phosphate), K₂HPO₄, is an amorphous white salt which is readily soluble in water.

Trisodium phosphate, tertiary sodium phosphate, Na₃PO₄, are colorless crystals which have a density of 1.62 gcm⁻³ and a melting point of 73-76° C. (decomposition) in the form of the dodecahydrate, have a melting point of 100° C. in the form of the decahydrate (corresponding to 19-20% P₂O₅), and have a density of 2.536 gcm⁻³ in anhydrous form (corresponding to 39-40% P₂O₅). Trisodium phosphate is readily soluble in water, with an alkaline reaction, and is prepared by evaporatively concentrating a solution of precisely 1 mol of disodium phosphate and 1 mol of NaOH. Tripotassium phosphate (tertiary or tribasic potassium phosphate), K₃PO₄, is a white, deliquescent, granular powder of density 2.56 gcm⁻³, has a melting point of 1,340° and is readily soluble in water with an alkaline reaction. It is formed, for example, when Thomas slag is heated with charcoal and potassium sulfate. Despite the relatively high cost, the more readily soluble and therefore highly active potassium phosphates are frequently preferred in the cleaning composition industry over corresponding sodium compounds.

Tetrasodium diphosphate (sodium pyrophosphate), Na₄P₂O₇, exists in anhydrous form (density 2.534 gcm⁻³, melting point 988 °, 880° also reported) and in the form of the decahydrate (density 1.815-1.836 gcm⁻³, melting point 94° with loss of water). Both substances are colorless crystals which dissolve in water with an alkaline reaction. Na₄P₂O₇ is formed when disodium phosphate is heated to >200° or by reacting phosphoric acid with sodium carbonate in the stoichiometric ratio and dewatering the solution by spraying. The decahydrate complexes heavy metal salts and hardness formers and therefore reduces the hardness of water. Potassium diphosphate (potassium pyrophosphate), K₄P₂O₇, exists in the form of the trihydrate and is a colorless, hygroscopic powder of density 2.33 gcm⁻³, which is soluble in water, the pH of the 1% solution at 25° being 10.4.

Condensation of NaH₂PO₄ or of KH₂PO₄ gives rise to higher molecular weight sodium phosphates and potassium phosphates, for which a distinction can be drawn between cyclic representatives, the sodium metaphosphates and potassium metaphosphates, and catenated types, the sodium polyphosphates and potassium polyphosphates. For the latter in particular, a multitude of names are in use: fused or calcined phosphates, Graham salt, Kurrol salt and Maddrell salt. All higher sodium and potassium phosphates are referred to collectively as condensed phosphates.

The industrially important pentasodium triphosphate, Na₅P₃O₁₀ (sodium tripolyphosphate), is a nonhygroscopic, white, water-soluble salt which is anhydrous or crystallizes with 6H₂O and has the general formula NaO—[P(O)(ONa)—O]ₙ—Na where n=3. About 17 g of the salt which is free of water of crystallization dissolve in 100 g of water at room temperature, at 60° approximately 20 g, at 100° around 32 g; after the solution has been heated at 100° for two hours, hydrolysis forms about 8% orthophosphate and 15% diphosphate. In the preparation of pentasodium triphosphate, phosphoric acid is reacted with sodium carbonate solution or sodium hydroxide solution in the stoichiometric ratio and the solution is dewatered by spraying. In a similar way to Graham salt and sodium diphosphate, pentasodium triphosphate dissolves many insoluble metal compounds (including lime soaps etc.). Pentapotassium triphosphate, K₅P₃O₁₀ (potassium tripolyphosphate), is available commercially, for example, in the form of a 50% by weight solution (>23% P₂O₅, 25% K₂O). The potassium polyphosphates find wide use in the washing and cleaning composition industry. There also exist sodium potassium tripolyphosphates which can likewise be used in the context of the present invention. They are formed, for example, when sodium trimetaphosphate is hydrolyzed with KOH:
(NaPO₃)₃+2 KOH→Na₃K₂P₃O₁₀+H₂O.

They can be used in accordance with the invention in precisely the same way as sodium tripolyphosphate, potassium tripolyphosphate or mixtures of the two; mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate can also be used in accordance with the invention.

Compositions preferred in the context of the present invention are characterized in that they comprise phosphate(s), preferably alkali metal phosphate(s), more preferably pentasodium triphosphate or pentapotassium triphosphate (sodium tripolyphosphate or potassium tripolyphosphate), in amounts of from 5 to 80% by weight, preferably from 15 to 75% by weight and in particular, from 20 to 70% by weight, based in each case on the weight of the washing or cleaning composition.

Particular preference is given to those inventive compositions in which the weight ratio of potassium tripolyphosphate to sodium tripolyphosphate present in the composition is more than 1:1, preferably more than 2:1, preferentially more than 5:1, more preferably more than 10:1 and especially more than 20:1. Particular preference is given to inventive compositions which comprise exclusively potassium tripolyphosphate.

Further builders are the alkali carriers. Alkali carriers include, for example, alkali metal hydroxides, alkali metal carbonates, alkali metal hydrogencarbonates, alkali metal sesquicarbonates, the aforementioned alkali metal silicates, alkali metal metasilicates and mixtures of the aforementioned substances, preference being given in the context of this invention to using the alkali metal carbonates, especially sodium carbonate, sodium hydrogencarbonate or sodium sesquicarbonate. Particular preference is given to a builder system comprising a mixture of tripolyphosphate and sodium carbonate. Particular preference is likewise given to a builder system comprising a mixture of tripolyphosphate and sodium carbonate and sodium disilicate.

Particularly preferred washing and cleaning compositions comprise carbonate(s) and/or hydrogencarbonate(s), preferably alkali metal carbonates, more preferably sodium carbonate, in amounts of from 2 to 50% by weight, preferably from 5 to 40% by weight and in particular, from 7.5 to 30% by weight, based in each case on the weight of the washing or cleaning composition.

The organic cobuilders which may be used in the inventive washing and cleaning compositions are in particular, polycarboxylates/polycarboxylic acids, polymeric polycarboxylates, aspartic acid, polyacetals, dextrins, further organic cobuilders (see below) and phosphonates. These substance classes are described below.

Organic builder substances which can be used are, for example, the polycarboxylic acids usable in the form of their sodium salts, polycarboxylic acids referring to those carboxylic acids which bear more than one acid function. Examples of these are citric acid, adipic acid, succinic acid, glutaric acid, malic acid, tartaric acid, maleic acid, fumaric acid, sugar acids, aminocarboxylic acids, nitrilotriacetic acid (NTA), as long as such a use is not objectionable on ecological grounds, and mixtures thereof. Preferred salts are the salts of the polycarboxylic acids such as citric acid, adipic acid, succinic acid, glutaric acid, tartaric acid, sugar acids and mixtures thereof.

The acids themselves may also be used. In addition to their builder action, the acids typically also have the property of an acidifying component and thus also serve to set a lower and milder pH of washing and cleaning compositions. In this connection, particular mention should be made of citric acid, succinic acid, glutaric acid, adipic acid, gluconic acid and any mixtures thereof.

Also suitable as builders are polymeric polycarboxylates; these are, for example, the alkali metal salts of polyacrylic acid or of polymethacrylic acid, for example, those having a relative molecular mass of from 500 to 70,000 g/mol.

In the context of this document, the molar masses specified for polymeric polycarboxylates are weight-average molar masses M_{w} of the particular acid form, which has always been determined by means of gel-permeation chromatography (GPC) using a UV detector. The measurement was against an external polyacrylic acid standard which, owing to its structural similarity to the polymers under investigation, provides realistic molecular weight values. These figures deviate considerably from the molecular weight data when polystyrenesulfonic acids are used as the standard. The molar masses measured against polystyrenesulfonic acids are generally distinctly higher than the molar masses specified in this document.

Suitable polymers are in particular, polyacrylates which preferably have a molecular mass of from 2,000 to 20,000 g/mol. Owing to their superior solubility, preference within this group may be given in turn to the short-chain polyacrylates which have molar masses of from 2,000 to 10,000 g/mol and more preferably from 3,000 to 5,000 g/mol.

Also suitable are copolymeric polycarboxylates, especially those of acrylic acid with methacrylic acid and of acrylic acid or methacrylic acid with maleic acid. Copolymers which have been found to be particularly suitable are those of acrylic acid with maleic acid which contain from 50 to 90% by weight of acrylic acid and from 50 to 10% by weight of maleic acid. Their relative molecular mass, based on free acids, is generally from 2,000 to 70,000 g/mol, preferably from 20,000 to 50,000 g/mol and in particular, from 30,000 to 40,000 g/mol.

Polyacrylates are commercially available, for example, under the names Versicol® E5, Versicol® E7 and Versicol® E9 (trademark of Allied Colloids), Narlex® LD 30 and Narlex® LD 34 (trademark of National Adhesives), Acrysol® LMW-10, Acrysol® LMW-20, Acrysol® LMW45 and Acrysol® A1-N (trademark of Rohm & Haas) and Sokalan® PA-20, Sokalan® PA-40, Sokalan® PA-70 and Sokalan® PA-110 (trademark of BASF). Ethylene/maleic acid copolymers are sold under the name EMA® (trademark of Monsanto), methyl vinyl ether/maleic acid copolymers under the name Gantrez® AN 119 (trademark of GAF Corp.) and acrylic acid/maleic acid copolymers under the name Sokalan® CP5 and Sokalan® CP7 (trademark of BASF). Acryloylphosphinates are obtainable as DKW® (trademark of National Adhesives) or Belperse® grades (trademark of Ciba-Geigy). In combination with the polymers mentioned or as the sole graying inhibitor, it is also possible to use graft copolymers which are obtained by grafting polyalkylene oxides having molecular weights between 2,000 and 100,000 with vinyl acetate. The acetate groups may optionally be up to 15% hydrolyzed. Polymers of this type are sold under the name Sokalan® HP22 (trademark of BASF).

The (co)polymeric polycarboxylates may be used either as a powder or as an aqueous solution. The content in the compositions of (co)polycarboxylates is preferably from 0.5 to 20% by weight, in particular, from 3 to 10% by weight.

To improve the water solubility, the polymers may also contain allylsulfonic acids, for example, allyloxybenzenesulfonic acid and methallylsulfonic acid, as monomers.

Also especially preferred are biodegradable polymers composed of more than two different monomer units, for example, those which contain, as monomers, salts of acrylic acid and of maleic acid, and vinyl alcohol or vinyl alcohol derivatives, or those which contain, as monomers, salts of acrylic acid and of 2-alkylallylsulfonic acid, and sugar derivatives.

Further preferred copolymers are those which preferably have, as monomers, acrolein and acrylic acid/acrylic acid salts or acrolein and vinyl acetate.

Further preferred builder substances which should likewise be mentioned are polymeric aminodicarboxylic acids, salts thereof or precursor substances thereof. Particular preference is given to polyaspartic acids or salts thereof.

Further suitable builder substances are polyacetals which can be obtained by reacting dialdehydes with polyolcarboxylic acids which have from 5 to 7 carbon atoms and at least 3 hydroxyl groups. Preferred polyacetals are obtained from dialdehydes such as glyoxal, glutaraldehyde, terephthalaldehyde, and mixtures thereof, and from polyolcarboxylic acids such as gluconic acid and/or glucoheptonic acid.

Further suitable organic builder substances are dextrins, for example, oligomers or polymers of carbohydrates, which can be obtained by partial hydrolysis of starches. The hydrolysis can be carried out by customary, for example, acid-catalyzed or enzyme-catalyzed, processes. The hydrolysis products preferably have average molar masses in the range from 400 to 500,000 g/mol. Preference is given to a polysaccharide having a dextrose equivalent (DE) in the range from 0.5 to 40, in particular, from 2 to 30, where DE is a common measure of the reducing action of a polysaccharide compared to dextrose, which has a DE of 100. It is also possible to use maltodextrins with a DE between 3 and 20 and dry glucose syrups with a DE between 20 and 37, and also what are known as yellow dextrins and white dextrins having relatively high molar masses in the range from 2,000 to 30,000 g/mol.

The oxidized derivatives of such dextrins are their reaction products with oxidizing agents which are capable of oxidizing at least one alcohol function of the saccharide ring to the carboxylic acid function.

Oxydisuccinates and other derivatives of disuccinates, preferably ethylenediaminedisuccinate, are also further suitable cobuilders. In this case, ethylenediamine-N,N′-disuccinate (EDDS) is preferably used in the form of its sodium or magnesium salts. Furthermore, in this connection, preference is also given to glyceryl disuccinates and glyceryl trisuccinates. Suitable use amounts in zeolite-containing and/or silicate-containing formulations are from 3 to 15% by weight.

Further organic cobuilders which can be used are, for example, acetylated hydroxycarboxylic acids or salts thereof, which may also be present in lactone form and which contain at least 4 carbon atoms and at least one hydroxyl group and a maximum of two acid groups.

A further class of substances having cobuilder properties is that of the phosphonates. These are in particular, hydroxyalkane- and aminoalkanephosphonates. Among the hydroxyalkanephosphonates, 1-hydroxyethane-1,1-diphosphonate (HEDP) is of particular significance as a cobuilder. It is preferably used in the form of the sodium salt, the disodium salt giving a neutral reaction and the tetrasodium salt an alkaline reaction (pH 9). Useful aminoalkanephosphonates are preferably ethylenediamine-tetramethylenephosphonate (EDTMP), diethylenetriaminepentamethylene-phosphonate (DTPMP) and higher homologs thereof. They are preferably used in the form of the neutrally reacting sodium salts, for example, as the hexasodium salt of EDTMP or as the hepta- and octasodium salt of DTPMP. From the class of the phosphonates, preference is given to using HEDP as a builder. In addition, the aminoalkanephosphonates have a marked heavy metal-binding capacity. Accordingly, especially when the compositions also comprise bleaches, it may be preferable to use aminoalkanephosphonates, especially DTPMP, or mixtures of the phosphonates mentioned.

In addition, it is possible to use all compounds which are capable of forming complexes with alkaline earth metal ions as cobuilders.

Inventive washing or cleaning compositions may also comprise washing- or cleaning-active polymers. The group of these polymers includes, for example, the rinse aid polymers and/or polymers effective as softeners. Washing or cleaning compositions preferred in accordance with the invention are characterized in that they, based on their total weight, contain from 0.1 to 50% by weight, preferably between 0.2 and 40% by weight, more preferably between 0.4 and 35% by weight and in particular, between 0.6 and 31% by weight of a polymer, preferably of at least one polymer from the group of the cationic, anionic or amphoteric polymers.

Polymers effective as softeners are, for example, the polymers containing sulfonic acid groups, which are used with particular preference.

Polymers which contain sulfonic acid groups and can be used with particular preference are copolymers of unsaturated carboxylic acids, monomers containing sulfonic acid groups and optionally further ionic or nonionogenic monomers.

In the context of the present invention, preferred monomers are unsaturated carboxylic acids of the formula XI
R¹(R²)C═C(R³)COOH (XI)
in which R¹ to R³ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms.

Among the unsaturated carboxylic acids which can be described by the formula X, preference is given in particular, to acrylic acid (R¹═R²═R³═H), methacrylic acid (R¹═R²═H; R³═CH₃) and/or maleic acid (R¹═COOH; R²═R³═H).

The monomers containing sulfonic acid groups are preferably those of the formula XII
R⁵(R⁶)C═C(R⁷)—X—SO₃H (XII)
in which R⁵ to R⁷ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms, and X is an optionally present spacer group which is selected from —(CH₂)ₙ—where n=from 0 to 4, —COO—(CH₂)ₖ— where k=from 1 to 6, —C(O)—NH—C(CH₃)₂— and —C(O)—NH—CH(CH₂CH₃)—.

Among these monomers, preference is given to those of the formulas XIIa, XIIb and/or XIIc
H₂C═CH—X—SO₃H (XIIa)
H₂C═C(CH₃)—X—SO₃H (XIIb)
HO₃S—X—(R⁶)C═C(R⁷)—X—SO₃H (XIIc)
in which R⁶ and R⁷ are each independently selected from —H, —CH₃, —CH₂CH₃, —CH₂CH₂CH₃, —CH(CH₃)₂ and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—.

Particularly preferred monomers containing sulfonic acid groups are 1-acrylamido-1-propanesulfonic acid (X=—C(O)NH—CH(CH₂CH₃) in formula XIIa), 2-acrylamido-2-propanesulfonic acid (X=—C(O)NH—C(CH₃)₂ in formula XIIa), 2-acrylamido-2-methyl-1-propanesulfonic acid (X=—C(O)NHCH(CH₃)CH₂— in formula XIIa), 2-methacrylamido-2-methyl-1-propanesulfonic acid (X=—C(O)NH—CH(CH₃)CH₂— in formula XIIb), 3-methacrylamido-2-hydroxypropanesulfonico acid (X=—C(O)NH—CH₂CH(OH)CH₂— in formula XIIb), allylsulfonic acid (X=CH₂ in formula XIIa), methallylsulfonic acid (X=CH₂ in formula XIIb), allyloxybenzenesulfonic acid (X=—CH₂—O—C₆H₄— in formula XIIa), methallyloxybenzenesulfonic acid (X=—CH₂—O—C₆H₄— in formula XIIb), 2-hydroxy-3-(2-propenyloxy)propanesulfonic acid, 2-methyl-2-propene-1-sulfonic acid (X=CH₂ in formula XIIb), styrenesulfonic acid (X=C₆H₄ in formula XIIa), vinylsulfonic acid (X not present in formula XIIa), 3-sulfopropyl acrylate (X=—C(O)NH—CH₂CH₂CH₂— in formula XIIa), 3-sulfopropyl methacrylate (X=—C(O)NH—CH₂CH₂CH₂— in formula XIIb), sulfomethacrylamide (X=—C(O)NH— in formula XIIb), sulfomethylmethacrylamide (X=—C(O)NH—CH₂— in formula XIIb) and water-soluble salts of the acids mentioned.

Useful further ionic or nonionogenic monomers are in particular, ethylenically unsaturated compounds. The content of monomers of group iii) in the polymers used in accordance with the invention is preferably less than 20% by weight, based on the polymer. Polymers to be used with particular preference consist only of monomers of groups i) and ii).

In summary, particularly preferred ingredients of the inventive washing or cleaning compositions are copolymers of
i) unsaturated carboxylic acids of the formula XI
   R¹(R²)C═C(R³)COOH (XI)
   in which R¹ to R³ are each independently-H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms,
ii) monomers of the formula XII containing sulfonic acid groups
   R⁵(R⁶)C═C(R⁷)—X—SO₃H (XII)
   in which R⁵ to R⁷ are each independently-H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms, and X is an optionally present spacer group which is selected from —(CH₂)ₙ — where n=from 0 to 4, —COO—(CH₂)ₖ— where k=from 1 to 6, —C(O)—NH—C(CH₃)₂— and —C(O)—NH—CH(CH₂CH₃)—
iii) optionally further ionic or nonionogenic monomers.

Particularly preferred copolymers consist of
i) one or more unsaturated carboxylic acids from the group of acrylic acid, methacrylic acid and/or maleic acid,
ii) one or more monomers containing sulfonic acid groups of the formulas XIIa, XIIb and/or XIIc:
   H₂C═CH—X—SO₃H (XIIa)
   H₂C═C(CH₃)—X—SO₃H (XIIb)
   HO₃S—X—(R⁶)C═C(R⁷)—X—SO₃H (XIIc)
   in which R¹ and R⁷ are each independently selected from —H, —CH₃, —CH₂CH₃, —CH₂CH₂CH₃, —CH(CH₃)₂ and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—
iii) optionally further ionic or nonionogenic monomers.

The copolymers may contain the monomers from groups i) and ii) and optionally iii) in varying amounts, and it is possible to combine any of the representatives from group i) with any of the representatives from group ii) and any of the representatives from group iii). Particularly preferred polymers have certain structural units which are described below.

Thus, preference is given, for example, to washing or cleaning compositions which are characterized in that they comprise one or more copolymers which contain structural units of the formula XIII
—[CH₂—CHCOOH]ₘ—[CH₂—CHC(O)—Y—SO₃H]ₚ— (XIII)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

These polymers are prepared by copolymerization of acrylic acid with an acrylic acid derivative containing sulfonic acid groups. Copolymerizing the acrylic acid derivative containing sulfonic acid groups with methacrylic acid leads to another polymer, the use of which in the inventive washing or cleaning compositions is likewise preferred and which is characterized in that the preferred washing or cleaning compositions comprise one or more copolymers which contain structural units of the formula XIV
—[CH₂—C(CH₃)COOH]ₘ—[CH₂—CHC(O)—Y—SO₃H]ₚ— (XIV)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

Acrylic acid and/or methacrylic acid can also be copolymerized entirely analogously with methacrylic acid derivatives containing sulfonic acid groups, which changes the structural units within the molecule. Thus, inventive washing or cleaning compositions which comprise one or more copolymers which contain structural units of the formula XV
—[CH₂—CHCOOH]ₘ—[CH₂—C(CH₃)C(O)—Y—SO₃H]ₚ— (XV)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—, are likewise a preferred embodiment of the present invention, just like preference is also given to washing or cleaning compositions which are characterized in that they comprise one or more copolymers which contain structural units of the formula XVI
—[CH₂—C(CH₃)COOH]ₘ—[CH₂—C(CH₃)C(O)—Y—SO₃H]ₚ— (XVI)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

Instead of acrylic acid and/or methacrylic acid, or in addition thereto, it is also possible to use maleic acid as a particularly preferred monomer from group i). This leads to washing or cleaning compositions which are preferred in accordance with the invention and are characterized in that they comprise one or more copolymers which contain structural units of the formula XVII
—[HOOCCH—CHCOOH]ₘ—[CH₂—CHC(O)—Y—SO₃H]ₚ— (XVII)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—, and to washing or cleaning compositions which are characterized in that they comprise one or more copolymers which contain structural units of the formula XVIII
—[HOOCCH—CHCOOH]ₘ—[CH₂—C(CH₃)C(O)O—Y—SO₃H]ₚ— (XVIII)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

In summary, preference is given to inventive washing or cleaning compositions which comprise one or more copolymers which contain structural units of the formulas XIII and/or XIV and/or XV and/or XVI and/or XVII and/or XVIII
—[CH₂—CHCOOH]ₘ—[CH₂—CHC(O)—Y—SO₃H]ₚ— (XIII)
—[CH₂—C(CH₃)COOH]ₘ—[CH₂—CHC(O)—Y—SO₃H]ₚ— (XIV)
—[CH₂—CHCOOH]ₘ—[CH₂—C(CH₃)C(O)—Y—SO₃H]ₚ— (XV)
—[CH₂—C(CH₃)COOH]ₘ—[CH₂—C(CH₃)C(O)—Y—SO₃H]ₚ— (XVI)
—[HOOCCH—CHCOOH]ₘ—[CH₂—CHC(O)—Y—SO₃H]ₚ— (XVII)
—[HOOCCH—CHCOOH]ₘ—[CH₂—C(CH₃)C(O)O—Y—SO₃H]ₚ— (XVIII)
in which m and p are each a whole natural number between 1 and 2,000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

In the polymers, all or some of the sulfonic acid groups may be in neutralized form, i.e. the acidic hydrogen atom of the sulfonic acid group may be replaced in some or all of the sulfonic acid groups by metal ions, preferably alkali metal ions and in particular, by sodium ions. Preference is given in accordance with the invention to washing or cleaning compositions which are characterized in that the sulfonic acid groups in the copolymer are present in partly or fully neutralized form.

The monomer distribution of the copolymers used in the inventive washing or cleaning compositions is, in the case of copolymers which contain only monomers from groups i) and ii), preferably in each case from 5 to 95% by weight of i) or ii), more preferably from 50 to 90% by weight of monomer from group i) and from 10 to 50% by weight of monomer from group ii), based in each case on the polymer.

In the case of terpolymers, particular preference is given to those which contain from 20 to 85% by weight of monomer from group i), from 10 to 60% by weight of monomer from group ii), and from 5 to 30% by weight of monomer from group iii).

The molar mass of the above-described sulfo copolymers used in the inventive washing or cleaning compositions can be varied in order to adapt the properties of the polymers to the desired end use. Preferred washing or cleaning compositions are characterized in that the copolymers have molar masses of from 2,000 to 200,000 gmol⁻¹, preferably from 4,000 to 25,000 gmol⁻¹ and in particular, from 5,000 to 15,000 gmol⁻¹.

Particularly preferred inventive washing or cleaning compositions have the feature that they contain at least one polymer containing sulfonic acid groups, preferably a copolymer composed of
i) unsaturated carboxylic acids
ii) monomers containing sulfonic acid groups
iii) optionally further ionic or nonionogenic monomers.

Preferred inventive compositions which are used as machine dishwasher detergents may further comprise amphoteric or cationic polymers to improve the rinse result. These particularly preferred polymers are characterized in that they have at least one positive charge. Such polymers are preferably water-soluble or water-dispersible, i.e. they have a solubility above 10 mg/ml in water at 25° C. Washing or cleaning compositions particularly preferred in the context of the present application are characterized in that they comprise at least one polymer which has a molecular weight above 2,000 and at least one positive charge.

Particularly preferred cationic or amphoteric polymers contain at least one ethylenically unsaturated monomer unit of the general formula
R¹(R²)C═C(R³)R⁴
in which R¹ to R⁴ are each independently —H, —CH₃, a straight-chain or branched, saturated alkyl radical having from 2 to 12 carbon atoms, a straight-chain or branched, mono- or polyunsaturated alkenyl radical having from 2 to 12 carbon atoms, alkyl or alkenyl radicals substituted by —NH₂, —OH or —COOH as defined above, a heteroatomic group having at least one positively charged group, a quaternized nitrogen atom or at least one amine group having a positive charge in the pH range between 2 and 11, or —COOH or —COOR⁵ where R⁵ is a saturated or unsaturated, straight-chain or branched hydrocarbon radical having from 1 to 12 carbon atoms.

Examples of the aforementioned (unpolymerized) monomer units are diallylamine, methyldiallylamine, dimethyldimethylammonium salts, acrylamidopropyl(trimethyl)ammonium salts (R¹, R² and R³=H, R⁴=C(O)NH(CH₂)₂N⁺(CH₃)₃X⁻), methacrylamidopropyl(trimethyl)ammonium salts (R¹ and R²=H, R³=CH₃, H, R⁴=C(O)NH(CH₂)₂N⁺(CH₃)₃X⁻).

Particular preference is given to using, as a constituent of the amphoteric polymers, unsaturated carboxylic acids of the general formula
R¹(R²)C═C(R³)COOH
in which R¹ to R³ are each independently —H, —CH₃, a straight-chain or branched, saturated alkyl radical having from 2 to 12 carbon atoms, a straight-chain or branched, mono- or polyunsaturated alkenyl radical having from 2 to 12 carbon atoms, alkyl or alkenyl radicals substituted by —NH₂, —OH or —COOH as defined above or —COOH or —COOR⁴ where R⁴ is a saturated or unsaturated, straight-chain or branched hydrocarbon radical having from 1 to 12 carbon atoms.

Particularly preferred amphoteric polymers contain, as monomer units, derivatives of diallylamine, in particular, dimethyldiallylammonium salt and/or methacrylamidopropyl(trimethyl)ammonium salt, preferably in the form of the chloride, bromide, iodide, hydroxide, phosphate, sulfate, hydrosulfate, ethylsulfate, methylsulfate, mesylate, tosylate, formate or acetate in combination with monomer units from the group of the ethylenically unsaturated carboxylic acids.

Before the compaction of the particulate premixture to washing or cleaning composition tablets, the premixture may be “powdered” with finely divided surface treatment compositions. This may be advantageous for the nature and physical properties both of the premixture (storage, compaction) and of the finished washing or cleaning composition tablets. Finely divided powdering compositions are known in the prior art, and usually zeolites, silicates or other inorganic salts are used. However, preference is given to “powdering” the premixture with finely divided zeolite, preference being given to zeolites of the faujasite type. In the context of the present invention, the term “zeolite of the faujasite type” indicates all three zeolites which form the faujasite subgroup of the zeolite structure group 4 (cf. Donald W. Breck: “Zeolite Molecular Sieves”, John Wiley & Sons, New York, London, Sydney, Toronto, 1974, page 92). In addition to the zeolite X, zeolite Y and faujasite and mixtures of these compounds can also be used, preference being given to the pure zeolite X.

Mixtures or cocrystals of zeolites of the faujasite type with other zeolites which do not necessarily have to belong to the zeolite structure group 4 can also be used as powdering compositions, but it is advantageous when at least 50% by weight of the powdering composition consists of a zeolite of the faujasite type.

In the context of the present invention, preference is given to washing and cleaning composition tablets which consist of a particulate premixture which comprises granular components and subsequently admixed pulverulent substances, the or one of the subsequently admixed pulverulent components being a zeolite of the faujasite type having particle sizes below 100 μm, preferably below 10 μm and in particular, below 5 μm, and making up at least 0.2% by weight, preferably at least 0.5% by weight and in particular, more than 1% by weight of the premixture to be compressed.

According to the invention, preference is given to washing and cleaning composition tablets which additionally comprise a disintegration assistant. Preference is also given to processes according to the invention in which the premixture additionally comprises a disintegration assistant, preferably a cellulose-based distintegration assistant, preferably in granular, cogranulated or compacted form, in amounts of from 0.5 to 10% by weight, preferably from 3 to 7% by weight and in particular, from 4 to 6% by weight, based in each case on the weight of the premixture. In addition to the surfactant, builder and disintegration assistant constituents mentioned, or in their stead, in the process according to the invention, the particulate premixtures to be compressed may additionally comprise one or more substances from the group of the bleaches, bleach activators, enzymes, pH modifiers, fragrances, perfume carriers, fluorescers, dyes, foam inhibitors, silicone oils, antiredeposition agents, optical brighteners, graying inhibitors, dye transfer inhibitors and corrosion inhibitors.

Among the compounds which serve as bleaches and supply H₂O₂ in water, sodium percarbonate, sodium perborate tetrahydrate and sodium perborate monohydrate are of particular significance. Further bleaches which can be used are, for example, peroxypyrophosphates, citrate perhydrates, and H₂O₂-supplying peracidic salts or peracids, such as perbenzoates, peroxophthalates, diperazelaic acid, phthaloimino peracid or diperdodecanedioic acid. Inventive compositions may also comprise bleaches from the group of organic bleaches. Typical organic bleaches are the diacyl peroxides, for example, dibenzoyl peroxide. Further typical organic bleaches are the peroxy acids, particular examples being the alkyl peroxy acids and the aryl peroxy acids. Preferred representatives are (a) the peroxybenzoic acid and ring-substituted derivatives thereof, such as alkylperoxybenzoic acids, but also peroxy-α-naphthoic acid and magnesium monoperphthalate, (b) the aliphatic or substituted aliphatic peroxy acids, such as peroxylauric acid, peroxystearic acid, ε-phthalimidoperoxycaproic acid [phthaloiminoperoxy-hexanoic acid (PAP.)], o-carboxybenzamido-peroxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyidiperoxybutane-1,4-dioic acid and N,N-terephthaloyldi(6-aminopercaproic acid) may be used.

The bleaches used in the inventive dispersions may also be substances which release chlorine or bromine. Among suitable chlorine- or bromine-releasing materials, useful examples include heterocyclic N-bromoamides and N-chloroamides, for example, trichloroisocyanuric acid, tribromoisocyanuric acid, dibromoisocyanuric acid and/or dichloroisocyanuric acid (DICA) and/or salts thereof with cations such as potassium and sodium. Hydantoin compounds, such as 1,3-dichloro-5,5-dimethylhydantoin, are likewise suitable.

Preferred inventive dispersions contain bleaches in amounts of from 1 to 40% by weight, preferably from 2.5 to 30% by weight and in particular, from 5 to 20% by weight, based in each case on the overall dispersion.

When the inventive compositions are used as machine dishwasher detergents, they may further comprise bleach activators as dispersed substances, in order to achieve improved bleaching action when cleaning at temperatures of 60° C. and below. Bleach activators which may be used are compounds which, under perhydrolysis conditions, give rise to aliphatic peroxocarboxylic acids having preferably from 1 to 10 carbon atoms, in particular, from 2 to 4 carbon atoms, and/or optionally substituted perbenzoic acid. Suitable substances bear O-acyl and/or N-acyl groups of the number of carbon atoms specified, and/or optionally substituted benzoyl groups. Preference is given to polyacylated alkylenediamines, in particular, tetraacetylethylenediamine (TAED), acylated triazine derivatives, in particular, 1,5-diacetyl-2,4-dioxohexahydro-1,3,5-triazine (DADHT), acylated glycolurils, in particular, tetraacetylglycoluril (TAGU), N-acylimides, in particular, N-nonanoylsuccinimide (NOSI), acylated phenolsulfonates, in particular, n-nonanoyl- or isononanoyloxybenzenesulfonate (n- or iso-NOBS), carboxylic anhydrides, in particular, phthalic anhydride, acylated polyhydric alcohols, in particular, triacetin, ethylene glycol diacetate and 2,5-diacetoxy-2,5-dihydrofuran.

Further bleach activators used with preference in the context of the present application are compounds from the group of the cationic nitriles, especially cationic nitrile of the formula 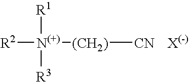
in which R¹ is —H, —CH₃, a C₂₋₂₄-alkyl or -alkenyl radical, a substituted C₂₋₂₄-alkyl or -alkenyl radical having at least one substituent from the group of —Cl, —Br, —OH, —NH₂, —CN, an alkyl- or alkenylaryl radical having a C₁₋₂₄-alkyl group, or is a substituted alkyl- or alkenylaryl radical having a C₁₋₂₄-alkyl group and at least one further substituent on the aromatic ring, R² and R³ are each independently selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂—CH₂—O)ₙH where n=1, 2, 3, 4, 5 or 6, and X is an anion.

Particularly preferred inventive compositions comprise a cationic nitrile of the formula 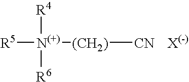
in which R⁴, R⁵ and R⁶ are each independently selected from —CH₃, —CH₂—CH₃, —CH₂—CH₂—CH₃, —CH(CH₃)—CH₃, where R⁴ may additionally also be —H, and X is an anion, it being preferred that R⁵=R⁶=—CH₃ and in particular, R⁴=R⁵=R⁶=—CH₃, and particular preference being given to compounds of the formulas (CH₃)₃N⁽⁺⁾CH₂—CNX⁻, (CH₃CH₂)₃N⁽⁺⁾CH₂—CNX⁻, (CH₃CH₂CH₂)₃N⁽⁺⁾CH₂—CNX⁻, (CH₃CH(CH₃))₃N⁽⁺⁾CH₂—CNX⁻ or (HO—CH₂—CH₂)₃N⁽⁺⁾CH₂—CNX⁻, particular preference being given in turn, from this group of substances, to the cationic nitrile of the formula (CH₃)₃N⁽⁺⁾CH₂—CN X⁻ in which X⁻ is an anion which is selected from the group of chloride, bromide, iodide, hydrogensulfate, methosulfate, p-toluenesulfonate (tosylate) or xylenesulfonate.

The bleach activators used may also be compounds which, under perhydrolysis conditions, give rise to aliphatic peroxocarboxylic acids having preferably from 1 to 10 carbon atoms, in particular, from 2 to 4 carbon atoms, and/or optionally substituted perbenzoic acid. Suitable substances bear O-acyl and/or N-acyl groups of the number of carbon atoms specified, and/or optionally substituted benzoyl groups. Preference is given to polyacylated alkylenediamines, in particular, tetraacetylethylenediamine (TAED), acylated triazine derivatives, in particular, 1,5-diacetyl-2,4-dioxohexahydro-1,3,5-triazine (DADHT), acylated glycolurils, in particular, tetraacetylglycoluril (TAGU), N-acylimides, in particular, N-nonanoylsuccinimide (NOSI), acylated phenolsulfonates, in particular, n-nonanoyl- or isononanoyloxybenzenesulfonate (n- or iso-NOBS), carboxylic anhydrides, in particular, phthalic anhydride, acylated polyhydric alcohols, in particular, triacetin, ethylene glycol diacetate, 2,5-diacetoxy-2,5-dihydrofuran, n-methylmorpholiniumacetonitrile methylsulfate (MMA), and also acetylated sorbitol and mannitol or mixtures thereof (SORMAN), acylated sugar derivatives, in particular, pentaacetylglucose (PAG), pentaacetylfructose, tetraacetylxylose and octaacetyllactose, and acetylated, optionally N-alkylated, glucamine and gluconolactone, and/or N-acylated lactams, for example, N-benzoylcaprolactam. Hydrophilically substituted acylacetals and acyllactams are likewise used with preference. Combinations of conventional bleach activators can also be used. The bleach activators are used in machine dishwasher detergents typically in amounts of from 0.1 to 20% by weight, preferably from 0.25 to 15% by weight and in particular, from 1 to 10% by weight, based in each case on the composition.

In addition to the conventional bleach activators, or instead of them, it is also possible to use so-called bleach catalysts in the composition. These substances are bleach-boosting transition metal salts or transition metal complexes, for example, salen or carbonyl complexes of Mn, Fe, Co, Ru or Mo. It is also possible to use complexes of Mn, Fe, Co, Ru, Mo, Ti, V and Cu with N-containing tripod ligands, and also Co-, Fe-, Cu- and Ru-ammine complexes as bleach catalysts.

When further bleach activators are to be used in addition to the nitrile quats, preference is given to using bleach activators from the group of the polyacylated alkylenediamines, in particular, tetraacetylethylenediamine (TAED), N-acylimides, in particular, N-nonanoylsuccinimide (NOSI), acylated phenolsulfonates, in particular, n-nonanoyl- or isononanoyloxybenzenesulfonate (n- or iso-NOBS), n-methylmorpholiniumacetonitrile methylsulfate (MMA), preferably in amounts up to 10% by weight, in particular, from 0.1% by weight to 8% by weight, particularly from 2 to 8% by weight and more preferably from 2 to 6% by weight, based in each case on the total weight of the dispersion.

Bleach-boosting transition metal complexes, in particular, with the central atoms Mn, Fe, Co, Cu, Mo, V, Ti and/or Ru, preferably selected from the group of manganese and/or cobalt salts and/or complexes, more preferably the cobalt (ammine) complexes, the cobalt (acetate) complexes, the cobalt (carbonyl) complexes, the chlorides of cobalt or manganese, and manganese sulfate, are used in customary amounts, preferably in an amount up to 5% by weight, in particular, from 0.0025% by weight to 1% by weight and more preferably from 0.01% by weight to 0.25% by weight, based in each case on the overall composition. In specific cases, though, it is also possible to use a greater amount of bleach activator.

A further important criterion for assessing a machine dishwasher detergent is, aside from its cleaning performance, the visual appearance of the dry dishes on completion of cleaning. Any calcium carbonate deposits which arise on dishes or in the interior of the machine might, for example, impair customer satisfaction and thus have a causal influence on the economic success of such a detergent. A further problem which has existed for some time in machine dishwashing is the corrosion of glassware, which can usually manifest itself by the appearance of clouding, smearing and scratches, but also by an iridescence of the glass surface. The observed effects are based essentially on two operations, the exit of alkali metal and alkaline earth metal ions from the glass in conjunction with a hydrolysis of the silicate network, and secondly in a deposition of silicatic compounds on the glass surface.

The problems mentioned can be solved using the inventive dispersions when, in addition to the aforementioned obligatory and any optional ingredients, certain glass corrosion inhibitors are incorporated into the compositions. Preferred inventive compositions therefore additionally comprise glass corrosion protectants, preferably from the group of magnesium and/or zinc salts and/or magnesium and/or zinc complexes.

A preferred class of compounds which can be added to the inventive compositions to prevent glass corrosion is that of insoluble zinc salts. These can position themselves during the dishwashing operation on the glass surface, where they prevent metal ions from the glass network from going into solution, and also the hydrolysis of the silicates. Additionally, these insoluble zinc salts also prevent the deposition of silicate on the surface of the glass, so that the glass is protected from the consequences outlined above.

In the context of this preferred embodiment, insoluble zinc salts are zinc salts which have a maximum solubility of 10 grams of zinc salt per liter of water at 20° C. Examples of insoluble zinc salts which are particularly preferred in accordance with the invention are zinc silicate, zinc carbonate, zinc oxide, basic zinc carbonate (Zn₂(OH)₂CO₃), zinc hydroxide, zinc oxalate, zinc monophosphate (Zn₃(PO₄)₂), and zinc pyrophosphate (Zn₂(P₂O₇)).

The zinc compounds mentioned are used in the inventive compositions preferably in amounts which bring about a content of zinc ions in the compositions of between 0.02 and 10% by weight, preferably between 0.1 and 5.0% by weight and in particular, between 0.2 and 1.0% by weight, based in each case on the composition. The exact content in the compositions of zinc salt or zinc salts is by its nature dependent on the type of the zinc salts—the less soluble the zinc salt used, the higher its concentration in the inventive compositions.

Since the insoluble zinc salts remain for the most part unchanged during the dishwashing operation, the particle size of the salts is a criterion to be considered, so that the salts do not adhere to glassware or parts of the machine. Preference is given here to inventive liquid aqueous machine dishwasher detergents in which the insoluble zinc salts have a particle size below 1.7 millimeters.

When the maximum particle size of the insoluble zinc salts is less than 1.7 mm, there is no risk of insoluble residues in the dishwasher. The insoluble zinc salt preferably has an average particle size which is distinctly below this value in order to further minimize the risk of insoluble residues, for example, an average particle size of less than 250 μm. The lower the solubility of the zinc salt, the more important this is. In addition, the glass corrosion-inhibiting effectiveness increases with decreasing particle size. In the case of very sparingly soluble zinc salts, the average particle size is preferably below 100 μm. For even more sparingly soluble salts, it may be lower still; for example, average particle sizes below 100 μm are preferred for the very sparingly soluble zinc oxide.

A further preferred class of compounds is that of magnesium and/or zinc salt(s) of at least one monomeric and/or polymeric organic acid. These have the effect that, even upon repeated use, the surfaces of glassware are not altered as a result of corrosion, and in particular, no clouding, smears or scratches, and also no iridescence of the glass surfaces, are caused.

Even though all magnesium and/or zinc salt(s) of monomeric and/or polymeric organic acids may be present in accordance with the invention in the claimed compositions, preference is given, as described above, to the magnesium and/or zinc salts of monomeric and/or polymeric organic acids from the groups of the unbranched, saturated or unsaturated monocarboxylic acids, the branched, saturated or unsaturated monocarboxylic acids, the saturated and unsaturated dicarboxylic acids, the aromatic mono-, di- and tricarboxylic acids, the sugar acids, the hydroxy acids, the oxo acids, the amino acids and/or the polymeric carboxylic acids. In the context of the present invention, preference is in turn given within these groups to the acids specified below:

From the group of unbranched, saturated or unsaturated monocarboxylic acids: methanoic acid (formic acid), ethanoic acid (acetic acid), propanoic acid (propionic acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid, dodecanoic acid (lauric acid), tridecanoic acid, tetradecanoic acid (myristic acid), pentadecanoic acid, hexadecanoic acid (palmitic acid), heptadecanoic acid (margaric acid), octadecanoic acid (stearic acid), eicosanoic acid (arachic acid), docosanoic acid (behenic acid), tetracosanoic acid (lignoceric acid), hexacosanoic acid (cerotic acid), triacotanoic acid (melissic acid), 9c-hexadecenoic acid (palmitoleic acid), 6c-octadecenoic acid (petroselic acid), 6t-octadecenoic acid (petroselaidic acid), 9c-octadecenoic acid (oleic acid), 9t-octadecenoic acid (elaidic acid), 9c,12c-octadecadienoic acid (linoleic acid), 9t,12t-octadecadienoic acid (linolaidic acid) and 9c, 12c, 15c-octadecatrienoic acid (linolenic acid).

From the group of branched, saturated or unsaturated monocarboxylic acids: 2-methylpentanoic acid, 2-ethylhexanoic acid, 2-propylheptanoic acid, 2-butyloctanoic acid, 2-pentylnonanoic acid, 2-hexyldecanoic acid, 2-heptylundecanoic acid, 2-octyldodecanoic acid, 2-nonyltridecanoic acid, 2-decyltetradecanoic acid, 2-undecylpentadecanoic acid, 2-dodecylhexadecanoic acid, 2-tridecylheptadecanoic acid, 2-tetradecyloctadecanoic acid, 2-pentadecylnonadecanoic acid, 2-hexadecyleicosanoic acid, 2-heptadecylheneicosanoic acid.

From the group of unbranched, saturated or unsaturated di- or tricarboxylic acids: propanedioic acid (malonic acid), butanedioic acid (succinic acid), pentanedioic acid (glutaric acid), hexanedioic acid (adipic acid), heptanedioic acid (pimelic acid), octanedioic acid (suberic acid), nonanedioic acid (azelaic acid), decanedioic acid (sebacic acid), 2c-butenedioic acid (maleic acid), 2t-butenedioic acid (fumaric acid), 2-butynedicarboxylic acid (acetylenedicarboxylic acid).

From the group of aromatic mono-, di- and tricarboxylic acids: benzoic acid, 2-carboxybenzoic acid (phthalic acid), 3-carboxybenzoic acid (isophthalic acid), 4-carboxybenzoic acid (terephthalic acid), 3,4-dicarboxybenzoic acid (trimellitic acid), 3,5-dicarboxybenzoic acid (trimesionic acid).

From the group of sugar acids: galactonic acid, mannonic acid, fructonic acid, arabinonic acid, xylonic acid, ribonic acid, 2-deoxyribonic acid, alginic acid.

From the group of hydroxy acids: hydroxyphenylacetic acid (mandelic acid), 2-hydroxypropionic acid (lactic acid), hydroxysuccinic acid (malic acid), 2,3-dihydroxybutanedioic acid (tartaric acid), 2-hydroxy-1,2,3-propanetricarboxylic acid (citric acid), ascorbic acid, 2-hydroxybenzoic acid (salicylic acid), 3,4,5-trihydroxybenzoic acid (gallic acid).

From the group of oxo acids: 2-oxopropionic acid (pyruvic acid), 4-oxopentanoic acid (levulinic acid).

From the group of amino acids: alanine, valine, leucine, isoleucine, proline, tryptophan, phenylalanine, methionine, glycine, serine, tyrosine, threonine, cysteine, asparagine, glutamine, aspartic acid, glutamic acid, lysine, arginine, histidine.

From the group of polymeric carboxylic acids: polyacrylic acid, polymethacrylic acid, alkylacrylamide/acrylic acid copolymers, alkylacrylamide/methacrylic acid copolymers, alkylacrylamide/methylmethacrylic acid copolymers, copolymers of unsaturated carboxylic acids, vinyl acetate/crotonic acid copolymers, vinylpyrrolidone/vinyl acrylate copolymers.

The spectrum of the zinc salts, preferred in accordance with the invention, of organic acids, preferably of organic carboxylic acids, ranges from salts which are sparingly soluble or insoluble in water, i.e. have a solubility below 100 mg/l, preferably below 10 mg/l, in particular, have zero solubility, to those salts which have a solubility in water above 100 mg/l, preferably above 500 mg/l, more preferably above 1 g/l and in particular, above 5 g/l (all solubilities at water temperature 20° C.). The first group of zinc salts includes, for example, zinc citrate, zinc oleate and zinc stearate; the group of soluble zinc salts includes, for example, zinc formate, zinc acetate, zinc lactate and zinc gluconate.

In a further preferred embodiment of the present invention, the compositions according to the invention comprise at least one zinc salt, but no magnesium salt of an organic acid, preferably at least one zinc salt of an organic carboxylic acid, more preferably a zinc salt from the group of zinc stearate, zinc oleate, zinc gluconate, zinc acetate, zinc lactate and/or zinc citrate. Preference is also given to zinc ricinoleate, zinc abietate and zinc oxalate.

A composition which is preferred in the context of the present invention contains zinc salt in amounts of from 0.1 to 5% by weight, preferably from 0.2 to 4% by weight and in particular, from 0.4 to 3% by weight, or zinc in oxidized form (calculated as Zn²⁺) in amounts of from 0.01 to 1% by weight, preferably from 0.02 to 0.5% by weight and in particular, from 0.04 to 0.2% by weight, based in each case on the total weight of the machine dishwasher detergent.

When the inventive tablets are used as dishwasher detergents, these cleaning compositions may comprise corrosion inhibitors to protect the ware or the machine, particularly silver protectants having particular significance in the field of machine dishwashing. It is possible to use the known substances from the prior art. In general, it is possible in particular, to use silver protectants selected from the group of the triazoles, the benzotriazoles, the bisbenzotriazoles, the aminotriazoles, the alkylaminotriazoles and the transition metal salts or complexes. Particular preference is given to using benzotriazole and/or alkylaminotriazole. Examples of the 3-amino-5-alkyl-1,2,4-triazoles to be used with preference in accordance with the invention include: 5-propyl-, -butyl-, -pentyl-, -heptyl-, -octyl-, -nonyl-, -decyl-, -undecyl-, -dodecyl-, -isononyl-, -Versatic-10 acid alkyl-, -phenyl-, -p-tolyl-, -(4-tert-butylphenyl)-, -(4-methoxyphenyl)-, -(2-,-3-, 4-pyridyl)-, -(2-thienyl)-, -(5-methyl-2-furyl)-, -(5-oxo-2-pyrrolidinyl)-3-amino-1,2,4-triazole. In machine dishwasher detergents, the alkylamino-1,2,4-triazoles or their physiologically compatible salts are used in a concentration of from 0.001 to 10% by weight, preferably from 0.0025 to 2% by weight, more preferably from 0.01 to 0.04% by weight. Preferred acids for the salt formation are hydrochloric acid, sulfuric acid, phosphoric acid, carbonic acid, sulfurous acid, organic carboxylic acids such as acetic acid, glycolic acid, citric acid, succinic acid. Very particularly effective are 5-pentyl-, 5-heptyl-, 5-nonyl-, 5-undecyl-, 5-isononyl-, 5-Versatic-10 acid alkyl-3-amino-1,2,4-triazoles, and also mixtures of these substances.

Frequently also found in cleaning formulations are active chlorine-containing agents which can significantly reduce the corrosion of the silver surface. In chlorine-free cleaners, particularly oxygen- and nitrogen-containing organic redox-active compounds, such as di- and trihydric phenols, for example, hydroquinone, pyrocatechol, hydroxyhydroquinone, gallic acid, phloroglucinol, pyrogallol and derivatives of these classes of compound are used. Salt- and complex-type inorganic compounds, such as salts of the metals Mn, Ti, Zr, Hf, V, Co and Ce, also frequently find use. Preference is given in this context to the transition metal salts which are selected from the group of manganese and/or cobalt salts and/or complexes, more preferably cobalt (ammine) complexes, cobalt (acetate) complexes, cobalt (carbonyl) complexes, the chlorides of cobalt or manganese, and manganese sulfate. Zinc compounds may likewise be used to prevent corrosion on the ware.

Instead of or in addition to the above-described silver protectants, for example, the benzotriazoles, it is possible to use redox-active substances in the inventive compositions. These substances are preferably inorganic redox-active substances from the group of the manganese, titanium, zirconium, hafnium, vanadium, cobalt and cerium salts and/or complexes, the metals preferably being in one of the oxidation states II, III, IV, V or VI.

The metal salts or metal complexes used should be at least partially soluble in water. The counterions suitable for the salt formation include all customary singly, doubly or triply negatively charged inorganic anions, for example, oxide, sulfate, nitrate, fluoride, but also organic anions, for example, stearate.

Metal complexes in the context of the invention are compounds which consist of a central atom and one or more ligands, and optionally additionally one or more of the above-mentioned anions. The central atom is one of the above-mentioned metals in one of the above-mentioned oxidation states. The ligands are neutral molecules or anions which are mono- or polydentate; the term “ligands” in the context of the invention is explained in more detail, for example, in Römpp Chemie Lexikon, Georg Thieme Verlag, Stuttgart/New York, 9th edition, 1990, page 2507. When the charge of the central atom and the charge of the ligand(s) within a metal complex do not add up to zero, depending on whether there is a cationic or an anionic charge excess, either one or more of the above-mentioned anions or one or more cations, for example, sodium, potassium, ammonium ions, ensure that the charge balances. Suitable complexing agents are, for example, citrate, acetyl acetonate or 1-hydroxyethane-1,1-diphosphonate.

The definition of “oxidation state” customary in chemistry is reproduced, for example, in Römpp Chemie Lexikon, Georg Thieme Verlag, Stuttgart/New York, 9th edition, 1991, page 3168.

Particularly preferred metal salts and/or metal complexes are selected from the group of MnSO₄, Mn(II) citrate, Mn(II) stearate, Mn(II) acetylacetonate, Mn(II) [1-hydroxyethane-1,1-diphosphonate], V₂O₅, V₂O₄, VO₂, TiOSO₄, K₂TiF₆, K₂ZrF₆, CoSO₄, Co(NO₃)₂, Ce(NO₃)₃, and mixtures thereof, so that preferred inventive machine dishwasher detergents are characterized in that the metal salts and/or metal complexes are selected from the group consisting of MnSO₄, Mn(II) citrate, Mn(II) stearate, Mn(II) acetylacetonate, Mn(II) [1-hydroxyethane-1,1-diphosphonate], V₂O₅, V₂O₄, VO₂, TiOSO₄, K₂TiF₆, K₂ZrF₆, CoSO₄, Co(NO₃)₂, Ce(NO₃)₃

These metal salts or metal complexes are generally commercial substances which can be used in the inventive compositions for the purposes of silver corrosion protection without prior cleaning. For example, the mixture of penta- and tetravalent vanadium (V₂O₅, VO₂, V₂O₄) known from the preparation of SO₃ (contact process) is therefore suitable, as is the titanyl sulfate, TiOSO₄, which is obtained by diluting a Ti(SO₄)₂ solution.

The inorganic redox-active substances, especially metal salts or metal complexes, are preferably coated, i.e. covered completely with a material which is water-tight, but slightly soluble at the cleaning temperatures, in order to prevent their premature disintegration or oxidation in the course of storage. Preferred coating materials which are applied by known methods, for instance by the melt coating method according to Sandwik from the foods industry, are paraffins, microcrystalline waxes, waxes of natural origin, such as carnauba wax, candelilla wax, beeswax, relatively high-melting alcohols, for example, hexadecanol, soaps or fatty acids. The coating material which is solid at room temperature is applied to the material to be coated in the molten state, for example, by centrifuging finely divided material to be coated in a continuous stream through a likewise continuously generated spray-mist zone of the molten coating material. The melting point has to be selected such that the coating material readily dissolves or rapidly melts during the silver treatment. The melting point should ideally be in the range between 45° C. and 65° C. and preferably in the 50° C. to 60° C. range.

The metal salts and/or metal complexes mentioned are present in the inventive compositions, especially machine dishwasher detergents, preferably in an amount of from 0.05 to 6% by weight, preferably from 0.2 to 2.5% by weight, based on the total weight of the composition.

To increase the washing or cleaning performance, inventive compositions may comprise enzymes, for which all enzymes established in the prior art for these purposes can be used in principle. These include in particular, proteases, amylases, lipases, hemicellulases, cellulases or oxidoreductases, and preferably mixtures thereof. These enzymes are in principle of natural origin; starting from the natural molecules, improved variants are available for use in washing and cleaning compositions and are preferably used accordingly. Inventive compositions contain enzymes preferably in total amounts of from 1×10⁻⁶ to 5 percent by weight based on active protein. The protein concentration may be determined with the aid of known methods, for example, the BCA method or the biuret method.

Among the proteases, preference is given to those of the subtilisin type. Examples thereof include the subtilisins BPN′ and Carlsberg, protease PB92, the subtilisins 147 and 309, Bacillus lentus alkaline protease, subtilisin DY and the enzymes thermitase and proteinase K which can be classified to the subtilases but no longer to the subtilisins in the narrower sense, and the proteases TW3 and TW7. The subtilisin Carlsberg is available in a developed form under the trade name Alcalase® from Novozymes A/S, Bagsvaerd, Denmark. The subtilisins 147 and 309 are sold under the trade names Esperase® and Savinase® respectively by Novozymes. The variants listed under the name BLAP® are derived from the protease of Bacillus lentus DSM 5483.

Further examples of useful proteases are the enzymes available under the trade names Durazym®, Relase®, Everlase®, Nafizym, Natalase®, Kannase® and Ovozymes® from Novozymes, those under the trade names Purafect®, Purafect® OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan and that under the name Proteinase K-16 from Kao Corp., Tokyo, Japan.

Examples of amylases which can be used in accordance with the invention are the α-amylases from Bacillus licheniformis, from B. amyloliquefaciens or from B. stearothermophilus and developments thereof which have been improved for use in washing and cleaning compositions. The B. licheniformis enzyme is available from Novozymes under the name Termamyl® and from Genencor under the name Purastar®ST. Development products of this α-amylase are obtainable from Novozymes under the trade names Duramyl® and Termamyl®ultra, from Genencor under the name Purastar®OxAm and from Daiwa Seiko Inc., Tokyo, Japan as Keistase®. The B. amyloliquefaciens α-amylase is sold by Novozymes under the name BAN®, and variants derived from the B. stearothermophilus α-amylase under the names BSG® and Novamyl®, likewise from Novozymes.

Enzymes which should additionally be emphasized for this purpose are the α-amylase from Bacillus sp. A 7-7 (DSM 12368), and the cyclodextrin glucanotransferase (CGTase) from B. agaradherens (DSM 9948).

Also suitable are the developments of α-amylase from Aspergillus niger and A. oryzae, which are available under the trade names Fungamyl® from Novozymes. Another commercial product is Amylase-LT®, for example.

Inventive compositions may comprise lipases or cutinases, especially owing to their triglyceride-cleaving activities, but also in order to generate peracids in situ from suitable precursors. Examples thereof include the lipases which were originally obtainable from Humicola lanuginosa (Thermomyces lanuginosus) or have been developed, in particular, those with the D96L amino acid substitution. They are sold, for example, under the trade names Lipolase®, Lipolase®Ultra, LipoPrime®, Lipozyme® and Lipex® from Novozymes. It is additionally possible, for example, to use the cutinases which have originally been isolated from Fusarium solani pisi and Humicola insolens. Lipases which are also useful can be obtained under the designations Lipase CE®, Lipase P®, Lipase B®, Lipase CES®, Lipase AKG®, Bacillis sp. Lipase®, Lipase AP®, Lipase M-AP® and Lipase AML® from Amano. Examples of lipases and cutinases from Genencor which can be used are those whose starting enzymes have originally been isolated from Pseudomonas mendocina and Fusarium solanii. Other important commercial products include the M1 Lipase® and Lipomax® preparations originally sold by Gist-Brocades and the enzymes sold under the names Lipase MY-30®, Lipase OF® and Lipase PL® by Meito Sangyo KK, Japan, and also the product Lumafast® from Genencor.

Inventive compositions may comprise further enzymes which are combined under the term hemicellulases. These include, for example, mannanases, xanthane lyases, pectin lyases (=pectinases), pectin esterases, pectate lyases, xyloglucanases (=xylanases), pullulanases and β-glucanases. Suitable mannanases are available, for example, under the names Gamanase® and Pektinex AR® from Novozymes, under the name Rohapec® B1 L from AB Enzymes and under the name Pyrolase® from Diversa Corp., San Diego, Calif., USA. The β-glucanase obtained from B. subtilis is available under the name Cereflo® from Novozymes.

To enhance the bleaching action, inventive washing and cleaning compositions may comprise oxidoreductases, for example, oxidases, oxygenases, catalases, peroxidases, such as haloperoxidases, chloroperoxidases, bromoperoxidases, lignin peroxidases, glucose peroxidases or manganese peroxidases, dioxygenases or laccases (phenol oxidases, polyphenol oxidases). Suitable commercial products include Denilite®1 and 2 from Novozymes. Advantageously, preferably organic, more preferably aromatic, compounds which interact with the enzymes are additionally added in order to enhance the activity of the oxidoreductases concerned (enhancers), or to ensure the electron flux in the event of large differences in the redox potentials of the oxidizing enzymes and the soilings (mediators).

The enzymes used in the inventive compositions derive, for example, either originally from microorganisms, for example, of the genera Bacillus, Streptomyces, Humicola, or Pseudomonas, and/or are produced in biotechnology processes known per se by suitable microorganisms, for instance by transgenic expression hosts of the genera Bacillus or filamentous fungi.

The enzymes in question are favorably purified via processes which are established per se, for example, via precipitation, sedimentation, concentration, filtration of the liquid phases, microfiltration, ultrafiltration, the action of chemicals, deodorization or suitable combinations of these steps.

The enzymes may be added to inventive compositions in any form established in the prior art. These include, for example, the solid preparations obtained by granulation, extrusion or lyophilization, or, especially in the case of liquid or gel-form compositions, solutions of the enzymes, advantageously highly concentrated, low in water and/or admixed with stabilizers.

Alternatively, the enzymes may be encapsulated either for the solid or for the liquid administration form, for example, by spray-drying or extrusion of the enzyme solution together with a preferably natural polymer, or in the form of capsules, for example, those in which the enzymes are enclosed as in a solidified gel, or in those of the core-shell type, in which an enzyme-containing core is coated with a water-, air- and/or chemical-impermeable protective layer. It is possible in layers applied thereto to additionally apply further active ingredients, for example, stabilizers, emulsifiers, pigments, bleaches or dyes. Such capsules are applied by methods known per se, for example, by agitated or roll granulation or in fluidized bed processes. Advantageously, such granules, for example, as a result of application of polymeric film formers, are low-dusting and storage-stable owing to the coating.

It is also possible to formulate two or more enzymes together, so that a single granule has a plurality of enzyme activities.

A protein and/or enzyme present in an inventive composition may be protected, particularly during storage, from damage, for example, inactivation, denaturation or decay, for instance by physical influences, oxidation or proteolytic cleavage. When the proteins and/or enzymes are obtained microbially, particular preference is given to inhibiting proteolysis, especially when the compositions also comprise proteases. For this purpose, inventive compositions may comprise stabilizers; the provision of such compositions constitutes a preferred embodiment of the present invention.

One group of stabilizers is that of reversible protease inhibitors. Frequently, benzamidine hydrochloride, borax, boric acids, boronic acids or salts or esters thereof are used, and of these in particular, derivatives having aromatic groups, for example, ortho-substituted, meta-substituted and para-substituted phenylboronic acids, or the salts or esters thereof. Peptidic protease inhibitors which should be mentioned include ovomucoid and leupeptin; an additional option is the formation of fusion proteins of proteases and peptide inhibitors.

Further enzyme stabilizers are amino alcohols such as mono-, di-, triethanol- and -propanolamine and mixtures thereof, aliphatic carboxylic acids up to C₁₂, such as succinic acid, other dicarboxylic acids or salts of the acids mentioned. Terminally capped fatty acid amide alkoxylates are also suitable as stabilizers. Certain organic acids used as builders are additionally capable of stabilizing an enzyme present.

Lower aliphatic alcohols, but in particular, polyols, for example, glycerol, ethylene glycol, propylene glycol or sorbitol, are other frequently used enzyme stabilizers. Calcium salts are likewise used, for example, calcium acetate or calcium formate, as are magnesium salts.

Polyamide oligomers or polymeric compounds such as lignin, water-soluble vinyl copolymers or cellulose ethers, acrylic polymers and/or polyamides stabilize the enzyme preparation against influences including physical influences or pH fluctuations. Polyamine N-oxide-containing polymers act simultaneously as enzyme stabilizers. Other polymeric stabilizers are the linear C₈-C₁₈ polyoxyalkylenes. Alkylpolyglycosides can likewise stabilize the enzymatic components of the inventive composition and even increase their performance. Crosslinked N-containing compounds likewise act as enzyme stabilizers.

Reducing agents and antioxidants increase the stability of the enzymes against oxidative decay. An example of a sulfur-containing reducing agent is sodium sulfite.

Preference is given to using combinations of stabilizers, for example, of polyols, boric acid and/or borax, the combination of boric acid or borate, reducing salts and succinic acid or other dicarboxylic acids or the combination of boric acid or borate with polyols or polyamino compounds and with reducing salts. The action of peptide-aldehyde stabilizers is increased by the combination with boric acid and/or boric acid derivatives and polyols, and further enhanced by the additional use of divalent cations, for example, calcium ions.

Preferred inventive tablets are characterized in that they additionally comprise one or more enzymes and/or enzyme preparations, preferably solid protease preparations and/or amylase preparations, in amounts of from 0.1 to 5% by weight, preferably of from 0.2 to 4.5% by weight and in particular, from 0.4 to 4% by weight, based in each case on the overall composition.

Compositions preferred in accordance with the invention are characterized in that they, based on their total weight, contain at least 20% by weight, preferably at least 30% by weight, more preferably at least 40% by weight and in particular, at least 50% by weight of builders and/or bleaches and/or bleach activators and/or washing- or cleaning-active polymers and/or glass corrosion protectants and/or silver protectants and/or enzymes. Particularly preferred inventive compositions consist to an extent of at least 90% by weight, preferably at least 92% by weight, preferentially to an extent of at least 94% by weight, more preferably to an extent of at least 96% by weight, especially preferably to an extent of at least 98% by weight and most preferably to an extent of at least 99.5% by weight exclusively of builders and/or bleaches and/or bleach activators and/or washing- or cleaning-active polymers and/or glass corrosion protectants and/or silver protectants and/or enzymes.

Particular preference is given to inventive washing or cleaning compositions which, based on their total weight, contain between 0.04 and 18% by weight, preferably between 0.08 and 16% by weight and in particular, between 0.2 and 14% by weight of one or more substances from the group of silver protectants, glass protectants or enzymes.

In addition, the washing and cleaning composition tablets may also comprise components which have a positive influence on the ability of oil and grease to be washed out of textiles (known as soil repellents). This effect becomes particularly clear when a textile is soiled which has already been washed before repeatedly with composition which has been produced in accordance with the invention and comprises this oil- and grease-dissolving component. The preferred oil- and grease-dissolving components include, for example, nonionic cellulose ethers such as methylcellulose and methylhydroxypropylcellulose with a proportion of methoxy groups of from 15 to 30% by weight and of hydroxypropoxy groups of from 1 to 15% by weight, based in each case on the nonionic cellulose ethers, and the polymers of phthalic acid and/or of terephthalic acid which are known from the prior art or of derivatives thereof, in particular, polymers of ethylene terephthalates and/or polyethylene glycol terephthalates or anionically and/or nonionically modified derivatives thereof. Among these, particular preference is given to the sulfonated derivatives of phthalic acid and of terephthalic acid polymers.

As optical brighteners, the tablets may comprise derivatives of diaminostilbenedisulfonic acid or alkali metal salts thereof. Suitable optical brighteners are, for example, salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, an anilino group or a 2-methoxyethylamino group. In addition, brighteners of the substituted diphenylstyryl type may be present, for example, the alkali metal salts of 4,4′-bis(2-sulfostyryl)diphenyl, of 4,4′-bis(4-chloro-3-sulfostyryl)diphenyl or of 4-(4-chlorostyryl)-4′-(2-sulfo-styryl)diphenyl. It is also possible to use mixtures of the aforementioned brighteners.

Dyes and fragrances are added to the washing and cleaning composition tablets produced in accordance with the invention in order to improve the esthetic impression of the products and to provide the consumer with not only the softness performance, but also with a visually and sensorily “typical and unmistakable” product. The perfume oils and/or fragrances used may be individual odorant compounds, for example, the synthetic products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether; the aldehydes include, for example, the linear alkanals having 8-18 carbon atoms, citral, citronellal, citronellyloxyacetaldehyde, cyclamen aldehyde, hydroxycitronellal, lilial and bourgeonal; the ketones include, for example, the ionones, α-isomethylionone and methyl cedryl ketone; the alcohols include anethole, citronellol, eugenol, geraniol, linalool, phenylethyl alcohol and terpineol; the hydrocarbons include primarily the terpenes such as limonene and pinene. However, preference is given to using mixtures of different odorants which together produce a pleasing fragrance note. Such perfume oils may also comprise natural odorant mixtures, as are obtainable from vegetable sources, for example, pine oil, citrus oil, jasmine oil, patchouli oil, rose oil or ylang-ylang oil. Likewise suitable are muscatel, sage oil, camomile oil, clove oil, balm oil, mint oil, cinnamon leaf oil, lime blossom oil, juniperberry oil, vetiver oil, olibanum oil, galbanum oil and labdanum oil, and also orange blossom oil, neroli oil, orange peel oil and sandalwood oil. Typically, the content in the washing and cleaning composition tablets produced in accordance with the invention of dyes is below 0.01% by weight, while fragrances may make up to 2% by weight of the overall formulation.

The fragrances can be incorporated directly into the compositions produced in accordance with the invention, but it may also be advantageous to apply the fragrances to carriers which intensify the adhesion of the perfume to the laundry and ensure long-lasting fragrance of the textiles by slower fragrance release. Useful such carrier materials have been found to be, for example, cyclodextrins, and the cyclodextrin-perfume complexes may additionally also be coated with further assistants.

In order to improve the esthetic impression of the compositions produced in accordance with the invention, they may be colored with suitable dyes. Preferred dyes, whose selection presents no difficulty at all to the person skilled in the art, have high storage stability and insensitivity toward the other ingredients of the products and to light, and also have no pronounced substantivity toward textile fibers, so as not to stain them.

1. A tablet comprising a compressed particulate washing or cleaning composition, wherein the tablet has, on its upper side, at least two reinforcement depressions whose horizontal dimension at the level of the tablet surface is greater than the depth of the reinforcement depression.

2. The tablet as claimed in claim 1, wherein the ratio of the horizontal dimension of the reinforcement depressions at the level of the tablet surface to the depth of the reinforcement depressions is from 1.01 to 5.

3. The tablet as claimed in claim 2, wherein the ratio of the horizontal dimension of the reinforcement depressions at the level of the tablet surface to the depth of the reinforcement depressions is from 1.05 to 2.

4. The tablet as claimed in claim 1, wherein the ratio of the depth of the reinforcement depressions to the tablet height is from 0.05 to 0.5.

5. The tablet as claimed in claim 4, wherein the ratio of the depth of the reinforcement depressions to the tablet height is from 0.15 to 0.3.

6. The tablet as claimed in claim 1, wherein the tablet has at least three reinforcement depressions.

7. The tablet as claimed in claim 6, wherein the tablet has at least seven reinforcement depressions.

8. The tablet as claimed in claim 1, wherein the reinforcement depressions run parallel to one another and to the tablet width.

9. The tablet as claimed in claim 1, wherein the reinforcement depressions run parallel to the tablet width and tablet length.

10. The tablet as claimed in claim 1, wherein the reinforcement depressions have the form of concentric circles or ellipses.

11. The tablet as claimed in claim 1, wherein the cross-section of the reinforcement depressions is triangular or semi-circular.

12. The tablet as claimed in claim 1, wherein the height of the tablet is from 5 to 25 mm.

13. The tablet as claimed in claim 1, wherein the depth of the reinforcement depressions is from 0.5 to 10 mm.

14. A tablet comprising a compressed particulate washing or cleaning composition characterized in that the tablet has on its upper side, reinforcement depressions radiating from a common center.

15. The tablet as claimed in claim 14, wherein the tablet has additional depressions that incorporate compositions other than washing or cleaning compositions.

16. The tablet as claimed in claim 15, wherein the additional depressions are present in the form of wavy lines.

17. A process for producing tablets of compressed particulate washing or cleaning composition, characterized in that the compression is effected by using an upper punch which has, on its pressing surface, at least two elevations for pressing of reinforcement depressions, whose horizontal dimension at the level of the pressing surface is greater than the height of the elevations.

18. The process as claimed in claim 17, wherein the upper punch has an adhesion-resistant coating.

19. The process as claim in claim 18, wherein the coating is selected from the group consisting of nickel-containing surface coatings in which ultra-fine polytetrafluoroethylene particles are enclosed, nickel-phosphorous alloy, and graphite-containing diamond particles.

20. The process according to claim 17, in which the coating of the elevations for the reinforcement depressions is hard and resistant toward high surface stresses and has a friction reducing and lubricating property.
